(19) United States
(12) Patent Application Publication
(10) Pub. No.: US 2021/0395829 A1
(43) Pub. Date: Dec. 23, 2021

(54) METHODS FOR MONITORING RESPONSE TO TREATMENT

(71) Applicant: ALFRED HEALTH, Melbourne, Victoria (AU)

(72) Inventors: ANDREW SPENCER, South Yarra, Victoria (AU); SRIDURGA MITHRAPRABHU, Endeavour Hills, Victoria (AU)

(73) Assignee: ALFRED HEALTH, Melbourne, Victoria (AU)

(21) Appl. No.: 17/279,574

(22) PCT Filed: Oct. 4, 2019
(86) PCT No.: PCT/AU2019/051077

§ 371 Date: Mar. 24, 2021

(30) Foreign Application Priority Data
Oct. 4, 2018 (AU) 2018903749

(51) Int. Cl. C12Q 1/6886

(52) U.S. Cl. CPC C12Q 1/6886; C12Q 2600/158; C12Q 2600/106

(57) ABSTRACT

The present invention relates to methods for monitoring the response of an individual to a treatment for multiple myeloma, and methods for treating an individual for multiple myeloma. More specifically, the methods include determining the expression of a gene that is regulated by a treatment for multiple myeloma and comparing the exRNA levels of the gene in a test sample to the exRNA levels in a control profile, wherein a change in the expression of the gene in the test sample compared to the control indicates that the individual has responded to the treatment.

### FIELD OF THE INVENTION

The present invention relates to methods and kits for monitoring or determining the efficacy of treatment for a myeloma, and methods of treating an individual for multiple myeloma.

### RELATED APPLICATION

The present application claims priority from Australian provisional application AU 2018903749, the entire contents of which are hereby incorporated by reference.

### BACKGROUND OF THE INVENTION

Multiple myeloma (MM) is an incurable haematological malignancy characterised by multi-focal tumour deposits throughout the bone marrow (BM). During disease progression, clonal plasma cells evolve the capacity to grow independently of the BM milieu and thus proliferate outside of the BM, manifesting as extramedullary multiple myeloma and/or plasma cell leukaemia.

Karyotypic instability and numeric chromosome abnormalities are present in virtually all MM. Primary translocations involving the immunoglobin (IgH) gene and FGFR3/MMSET, CCND1, CCND3, or MAF occur during the disease pathogenesis and secondary translocation involving the MYC gene occurs during disease progression.

Treatment of MM has witnessed significant progress with the implementation of proteasome inhibitors and immunomodulatory agents, however, the disease remains incurable with cells acquiring resistance to systemic therapies through accumulation of mutations that are often not present during the initial stages of the disease. Resistance to therapy is often mediated through genetic evolution of the MM cells, with the more resistant clones possessing a growth and survival advantage.

Current practice for diagnosis and prediction of prognosis is to perform sequential BM biopsies but the genetic information obtained from biopsies is confounded by the known inter and intra-clonal heterogeneity of the tumour(s).

There exists a need for improved or alternative methods for determining diagnosis, prediction of prognosis of multiple myeloma and/or monitoring efficacy of treatment.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a method of determining the likelihood of success of a treatment for multiple myeloma in an individual, the method comprising:
providing an individual who has received a treatment for multiple myeloma, wherein the treatment includes lenalidomide;
determining the levels of exRNA from one or more of cereblon, ikaros and aiolos in a test sample comprising extracellular RNA (exRNA) from the individual;
comparing the levels of exRNA from the one or more of cereblon, ikaros and aiolos in the test sample to a control profile representative of exRNA in a multiple myeloma patient prior to treatment for multiple myeloma;
determining that the treatment has likely not been successful when the levels of exRNA from one or more of cereblon, ikaros and aiolos in the test sample stays the same or does not increase in response to the treatment with lenalidomide.

The present invention provides a method of determining the likelihood of success of a treatment for multiple myeloma in an individual, the method comprising:
providing an individual who has received a treatment for multiple myeloma, wherein the treatment includes lenalidomide;
determining the levels of exRNA from one or more of cereblon, ikaros and aiolos in a test sample of extracellular RNA (exRNA) from the individual;
comparing the levels of the one or more of cereblon, ikaros and aiolos in the test sample to a control profile representative of exRNA in a multiple myeloma patient prior to treatment for multiple myeloma;
determining that the treatment has likely been successful when the levels of exRNA from one or more of cereblon, ikaros and aiolos in the test sample increases in response to the treatment with lenalidomide.

The present invention also provides a method for determining an early response in an individual to a treatment for multiple myeloma, the method comprising:
providing an individual who has received a treatment for multiple myeloma, wherein the treatment includes lenalidomide;
determining the levels of exRNA from one or more of cereblon, ikaros and aiolos, in a test sample comprising extracellular RNA (exRNA) obtained from the individual at a time point of no more than 20 days after then commencement of the treatment;
comparing the levels of exRNA from the one or more of cereblon, ikaros and aiolos in the test sample to a control profile representative of exRNA in a multiple myeloma patient prior to treatment for multiple myeloma;

wherein an increase in the levels of one or more of cereblon, ikaros and aiolos in the test sample compared to the control indicates that the individual has responded to the treatment. Preferably, the test sample is obtained fewer than 15 or 10 days, and more preferably 5 days or less since commencement of the treatment.

The present invention also provides a method for predicting the likelihood of overall survival of an individual who has received a treatment for multiple myeloma, the method comprising:
determining the levels of exRNA from one or more of cereblon, ikaros and aiolos in a test sample comprising exRNA obtained from the individual after receiving the treatment;
comparing the level of exRNA from the one or more of cereblon, ikaros and aiolos, in the test sample to a control profile representative of exRNA in a multiple myeloma patient prior to treatment for multiple myeloma;

wherein an increase in the level of exRNA from the one or more of cereblon, ikaros and aiolos in the test sample compared to the control correlates with an increased probability of recurrence free survival or overall survival of the subject and a later time,

thereby predicting the likelihood of overall survival of the individual.

The invention also provides a method for predicting the likelihood of overall survival of an individual who has received a treatment for multiple myeloma, the method comprising:
determining the levels of exRNA from the one or more of cereblon, ikaros and aiolos in a test sample comprising exRNA obtained from the individual after receiving the treatment;
comparing the level of exRNA from the one or more of cereblon, ikaros and aiolos in the test sample to a control profile representative of exRNA in a multiple myeloma patient prior to treatment for multiple myeloma;
wherein a decrease in the level of one or more of cereblon, ikaros and aiolos from the gene in the test sample compared to the control correlates with an decreased probability of recurrence free survival or overall survival of the subject and a later time,

thereby predicting a low likelihood of overall survival of the individual.

The present invention provides a method for providing a prognosis of an individual having multiple myeloma responding to a treatment regime, the method comprising:
determining the levels of exRNA from one or more of cereblon, ikaros and aiolos in a test sample comprising exRNA obtained from the individual after receiving the treatment;
comparing the level of exRNA from the one or more of cereblon, ikaros and aiolos in the test sample to a control profile representative of exRNA in a multiple myeloma patient prior to receiving the treatment for multiple myeloma;
wherein an increase in the level of exRNA from one or more of cereblon, ikaros and aiolos in the test sample compared to the control is indicative of the individual's prognosis of responding to the treatment regimen,
thereby providing a prognosis that the individual is responding to the treatment regimen.

In any aspect or embodiment of the invention, a method may be used for providing a prognosis for recurrence free survival, overall survival, four year survival or other clinically or biochemically detectable response to a treatment regime.

In any aspect or embodiment of the invention described herein, a high likelihood of overall survival is at least 95%, 94%, 93%, 92%, 91%, 90%, 85%, 80%, 75% or 70%.

In any aspect or embodiment of the invention described herein, a low likelihood of overall survival is less than 60%, 55%, 54%, 53%, 52%, 51%, 50%, 49%, 48%, 47%, 46%, 45% or 44%.

In any aspect or embodiment of the invention, a method may be used for providing a prognosis for recurrence free survival, overall survival, four year survival or other clinically or biochemically detectable response to a treatment regime.

The present invention provides a method of treating an individual for multiple myeloma, the method comprising:
providing an individual who has received a treatment for multiple myeloma, wherein the treatment includes lenalidomide;
determining the expression of one or more of cereblon, ikaros and aiolos in a test sample comprising extracellular RNA (exRNA) from the individual;
comparing the expression of one or more of cereblon, ikaros and aiolos in the test sample to a control profile representative of exRNA in a multiple myeloma patient prior to treatment for multiple myeloma;
administering an alternative treatment to the individual when the expression of one or more of cereblon, ikaros and aiolos in the test sample decreases, stays the same or does not increase in response to the treatment.

The present invention provides a method of treating an individual for multiple myeloma, the method comprising:
providing an individual who has received a treatment for multiple myeloma, wherein the treatment includes lenalidomide;
determining the expression of one or more of cereblon, ikaros and aiolos in a test sample comprising extracellular RNA (exRNA) from the individual;
comparing the expression of one or more of cereblon, ikaros and aiolos in the test sample to a control profile representative of exRNA in a multiple myeloma patient prior to treatment for multiple myeloma;
continuing to administer the treatment when the expression of one or more of cereblon, ikaros and aiolos in the test sample increases in response to the treatment.

The present invention provides a method of treating an individual with lenalidomide, the method comprising:
providing an individual with multiple myeloma, or suspected of having multiple myeloma;
administering a treatment for multiple myeloma to the individual, wherein the treatment includes lenalidomide;
determining the expression of one or more of cereblon, ikaros and aiolos in a test sample comprising extracellular RNA (exRNA) obtained from the individual after administration of the treatment;
comparing the expression of one or more of cereblon, ikaros and aiolos in the test sample to a control profile representative of exRNA in a multiple myeloma patient prior to treatment for multiple myeloma;
continuing to administer the treatment that includes lenalidomide when the expression of one or more of cereblon, ikaros and aiolos in the test sample increases in response to the treatment; or
administering an alternative treatment to the individual when the expression of one or more of cereblon, ikaros and aiolos in the test sample decreases, stays the same or does not increase in response to the treatment,

thereby treating the individual.

Preferably, the levels of exRNA from cereblon and ikaros are measured, and the treatment is continued when the levels of exRNA from both cereblon and ikaros increase, or alternatively, administering an alternative treatment to the individual when the levels of exRNA from both cereblon and ikaros decrease or stay the same.

In any embodiment, the expression or levels of exRNA for all three of cereblon, ikaros and aiolos increase, indicating that the individual has responded to the treatment, and that the treatment can be continued. Preferably, the expression of at least cereblon increases, more preferably, cereblon and ikaros levels increase.

In certain embodiments, the levels of exRNA from interferon regulatory factor 4 (IRF4) are also determined, and the treatment is continued when there is an increase in the levels of exRNA from ikaros and a decrease in the levels of exRNA from IRF4. Alternatively, when the levels of exRNA from IRF4 are also determined, an alternative treatment is administered when there is no change or a decrease in exRNA from ikaros and an increase or no change in the levels if IRF4.

When the levels of IRF4 are decreased in comparison to the test sample, this is indicative that the individual has responded to the treatment. Accordingly, in a preferred embodiment, the expression or levels of exRNA of all four of cereblon, ikaros, aiolos, and IRF4 are determined. When the expression of at least one of cereblon, ikaros and aiolos increases, and/or the expression of IRF4 decreases, this indicates that the individual has responded to the treatment and treatment can be continued. Preferably, the expression of at least cereblon increases, and at least IRF4 decreases

In any embodiment of the invention, the individual who has received treatment for multiple myeloma is an individual with relapsed and/or refractory multiple myeloma, including an individual who has not responded to a prior treatment. In certain embodiments, the prior treatment may be lenalidomide but not in combination with azacitidine or dexamethasone.

Preferably, the step of comparing the expression of the gene in the test sample to a control profile, and the determining to cease or continue the treatment is made within 20 days of the commencement of treatment. More preferably, the step of comparing is done fewer than 15, fewer than 10, fewer than 5 or fewer than 3 days following commencement of the treatment.

In any embodiment of the above invention, the method includes ceasing the initial treatment received by the individual when the individual does not respond to the treatment and commencing the individual on an alternative treatment.

In further aspects of the invention above, where the individual has not responded to the treatment administered, there comprises the step of administering one or more alternative drugs to treat the individual. Preferably, the treatment includes administering a drug or drugs which is/are different to that previously administered to the patient, such that the overall treatment of the individual for multiple myeloma is modified. In some embodiments, the drug or drugs that were previously administered to the patient is/are supplemented with one or more additional drugs. In alternative embodiments, the drug or drugs that were previously administered is/are replaced with one or more alternative drugs.

In any embodiment, administering an alternative treatment to the individual includes ceasing the administration of the first treatment. Alternatively, the alternative treatment can include additional drugs to supplement the first treatment.

In any embodiment of the invention, the methods include determining the expression of one or more additional genes expected to, or which are known to be regulated by the treatment.

In any embodiment, the test sample comprising exRNA, or test sample of exRNA, is any biological sample obtained from the individual that contains exRNA. In any embodiment, the step of providing a test sample of exRNA may involve obtaining a biological sample directly from the individual, and extracting the exRNA from the biological sample. The biological sample may be selected from: venous blood (peripheral blood), saliva, breast milk, urine, semen, menstrual blood, and vaginal fluid. Preferably, the biological sample containing exRNA is a sample of peripheral blood. Accordingly, in any embodiment, the step of providing a test sample of exRNA may include obtaining peripheral blood sample directly from the individual, and extracting the exRNA from the blood sample.

In any embodiment of the invention, the test sample may comprise, consist essentially of or consist of exRNA.

In any embodiment of the invention, a step of obtaining a test sample of peripheral blood may involve obtaining a peripheral blood sample directly from the individual.

In any embodiment, the control profile is any biological sample obtained from an individual that has multiple myeloma or has received a treatment for multiple myeloma, wherein the biological sample contains exRNA. The biological sample may be selected from: venous blood (peripheral blood), saliva, breast milk, urine, semen, menstrual blood, and vaginal fluid. Preferably, the biological sample containing exRNA is a sample of peripheral blood.

In any embodiment, the control biological sample containing exRNA is obtained from the individual prior to receiving treatment for multiple myeloma. In alternative embodiments, the control profile is obtained from a database comprising exRNA levels in a biological sample from one or more multiple myeloma patients, obtained prior to the patients receiving treatment for multiple myeloma.

Preferably the control profile is obtained 1, 2, 5, 10, 20, 30 or more days prior to the individual receiving treatment for multiple myeloma.

In any embodiment of the invention, the control sample may comprise, consist essentially of or consist of exRNA.

In any embodiment of the invention, determining the expression of a gene or comparing the expression levels of a gene can be using standard techniques, including quantitative RT-PCR and droplet digital PCR (ddPCR) to determine fold changes in expression relative to a control protein. Alternatively, determining or comparing gene expression includes determining the number of copies of the gene expressed per volume of the biological sample. In certain embodiments, determining the expression or comparing the expression levels of a gene includes determining or comparing the copies of the gene per ml of peripheral blood sample.

It will be understood that determining the expression of a gene may involve detection of a portion or a fragment of an RNA derived from a gene, rather than the full length RNA transcript. In other words, the present invention contemplates the identification of an RNA molecule of sufficient length to confirm the expression of a transcript from a gene as described herein.

In any embodiment of the invention, a fold change in exRNA gene expression levels of at least 0.01, 0.05, 0.1, 0.5, 1, 2, or more in the direction expected (e.g., fold increase for positively regulated genes, and fold decrease for negatively regulated genes) can be interpreted as providing an indication that the individual has responded to the treatment.

The present invention provides a method for monitoring the response of an individual to a treatment for multiple myeloma, the method comprising:
providing an individual who has received a treatment for multiple myeloma, wherein the treatment includes an IMid;
determining the expression of one or more of the genes cereblon, ikaros and aiolos in a test sample of extracellular RNA (exRNA) from the individual;
comparing the expression of the one or more of cereblon, ikaros and aiolos in the test sample to a control profile representative of exRNA in a multiple myeloma patient prior to treatment for multiple myeloma;

wherein an increase in the expression of one or more of cereblon, ikaros and aiolos in the test sample compared to the control indicates that the individual has responded to the treatment.

The present invention provides a method for predicting the likelihood that an individual has responded to a treatment for multiple myeloma, the method comprising:
providing an individual who has received a treatment for multiple myeloma, wherein the treatment includes an IMid;
determining the expression of one or more of the genes cereblon, ikaros and aiolos in a test sample of extracellular RNA (exRNA) from the individual;
comparing the expression of the one or more of cereblon, ikaros and aiolos in the test sample to a control profile representative of exRNA in a multiple myeloma patient prior to treatment for multiple myeloma;

wherein an increase in the expression of one or more of cereblon, ikaros and aiolos in the test sample compared to the control indicates that the individual has responded to the treatment.

The present invention provides a method for monitoring the response of an individual to a treatment for multiple myeloma, the method comprising:
providing a test sample of exRNA from an individual who has received a treatment for multiple myeloma, wherein the treatment includes an IMid;
determining the expression of one or more of the genes cereblon, ikaros and aiolos in the test sample;
providing a control profile containing data on the expression of exRNA from the genes cereblon, ikaros and aiolos in the individual prior to receiving the treatment;
comparing the expression of one or more of the genes cereblon, ikaros and aiolos in the test sample to the control profile;

wherein an increase in the expression of one or more of the genes cereblon, ikaros and aiolos in the test sample compared to the control indicates that the individual has responded to the treatment.

The present invention provides a method for determining an early response in an individual to a treatment for multiple myeloma, the method comprising:
determining the expression of one or more of the genes cereblon, ikaros and aiolos in a test sample of extracellular RNA (exRNA) from an individual who has received a treatment for multiple myeloma, wherein the treatment includes an Imid;
comparing the expression of one or more of cereblon, ikaros and aiolos in the test sample to a control profile representative of exRNA in a multiple myeloma patient prior to treatment for multiple myeloma;

wherein an increase in the expression of one or more of cereblon, ikaros and aiolos in the test sample compared to the control indicates that the individual has responded to the treatment. Preferably, the test sample is obtained fewer than 20 days after commencement of the treatment for multiple myeloma. More preferably, the test sample is obtained fewer than 15 or 10 days, and more preferably 5 days or less since commencement of the treatment.

Preferably the control profile is obtained 1, 2, 5, 10, 20, 30 or more days prior to the individual receiving treatment for multiple myeloma.

In any embodiment, the expression of at least cereblon and of ikaros or of cereblon and aiolos is determined. In any embodiment, the expression of all three of cereblon, ikaros, and aiolos is determined, and an increase in exRNA from all three genes indicates that the individual has responded to the treatment.

In one embodiment, the control profile is a sample of peripheral blood obtained from the individual prior to receiving treatment for multiple myeloma. In alternative embodiments, the control profile is obtained from a database comprising exRNA levels I peripheral blood from one or more multiple myeloma patients, obtained prior to the patients receiving treatment for multiple myeloma.

Preferably, the IMid is selected from lenalidomide, pomolidomide, thalidomide and apremilast.

In any embodiment, the treatment for multiple myeloma further includes a treatment with a hypomethylating agent. In one embodiment, the hypomethylating agent includes azacitidine.

Alternatively, the treatment for multiple myeloma is selected from: the combination of azacitidine and lenalidomide, or the combination of azacitidine, lenalidomide and dexamethasone.

In any embodiment of the invention, the individual who has received treatment for multiple myeloma is an individual with relapsed and/or refractory multiple myeloma, including an individual who has not responded to a prior treatment that included lenalidomide but not in combination with azacitidine or dexamethasone.

The present invention provides a method of treating an individual for multiple myeloma, the method comprising:
providing an individual who has received a treatment for multiple myeloma, wherein the treatment includes lenalidomide;
determining the levels of exRNA from one or more of cereblon, ikaros and aiolos in a test sample of extracellular RNA (exRNA) from the individual;
comparing the levels of exRNA from the one or more of cereblon, ikaros and aiolos in the test sample to a control profile representative of exRNA in a multiple myeloma patient prior to treatment for multiple myeloma;
administering an alternative treatment to the individual when the levels of exRNA from one or more of cereblon, ikaros and aiolos in the test sample stays the same or does not increase in response to the treatment with lenalidomide.

The present invention provides a method of treating an individual for multiple myeloma, the method comprising:
providing an individual who has received a treatment for multiple myeloma, wherein the treatment includes lenalidomide;
determining the levels of exRNA from one or more of cereblon, ikaros and aiolos in a test sample of extracellular RNA (exRNA) from the individual;
comparing the levels of the one or more of cereblon, ikaros and aiolos in the test sample to a control profile representative of exRNA in a multiple myeloma patient prior to treatment for multiple myeloma;
continuing to administer the treatment to the individual when the levels of exRNA from one or more of cereblon, ikaros and aiolos in the test sample increases in response to the treatment with lenalidomide.

Preferably, the levels of exRNA from cereblon and ikaros are measured, and the treatment is continued when the levels of exRNA from both cereblon and ikaros increase, or alternatively, administering an alternative treatment to the individual when the levels of exRNA from both cereblon and ikaros decrease or stay the same.

In certain embodiments, the levels of exRNA from interferon regulatory factor 4 (IRF4) are also determined, and the treatment is continued when there is an increase in the levels of exRNA from ikaros and a decrease in the levels of exRNA from IRF4. Alternatively, when the levels of exRNA from IRF4 are also determined, administering an alternative treatment when there is no change or a decrease in exRNA from ikaros and an increase or no change in the levels of exRNA from IRF4.

In certain embodiments, the levels of exRNA from TGFβ1 (transcription growth factor beta 1) are also determined, and the treatment is continued when there is an increase in the levels of exRNA from ikaros and an increase in the levels of exRNA from TGFβ1. Alternatively, when the levels of exRNA from TGFβ1 are also determined, an alternative treatment is administered when the is no change or a decrease in exRNA from ikaros and a decrease or no change in the levels of exRNA from TGFβ1.

The present invention provides a method of treating an individual for multiple myeloma, the method comprising:
providing an individual who has received a treatment for multiple myeloma, wherein the treatment includes lenalidomide;
determining the expression of a gene encoding a protein that is positively regulated by lenalidomide, in a test sample of extracellular RNA (exRNA) from the individual;
comparing the expression of the gene in the test sample to a control profile representative of exRNA in a multiple myeloma patient prior to treatment for multiple myeloma;
administering an alternative treatment to the individual when the expression of the gene in the test sample stays the same or does not decrease in response to the treatment with lenalidomide.

Preferably, a gene that encodes a protein that is activated by lenalidomide, or a gene which is positively regulated by lenalidomide is selected from: the group consisting of: cereblon, ikaros, aiolos, and TGFβ1.

Preferably, a gene that encodes a protein that is inhibited by lenalidomide or a gene which is negatively regulated by lenalidomide is IRF4.

The present invention provides use of an IMid in the manufacture of a medicament for the treatment of multiple myeloma in an individual, wherein the individual has been determined as likely to respond to treatment by any method of the invention described herein.

The present invention provides an IMid for use in the treatment of multiple myeloma in an individual, wherein the individual has been determined as likely to respond to treatment by any method of the invention described herein.

The present invention provides a method for determining the likelihood that an individual will respond to a treatment for multiple myeloma, wherein the treatment comprises an immunomodulatory imide (IMid) compound, the method comprising:
determining the expression of ikaros in a test sample comprising exRNA derived from both tumour and non-tumour cells from an individual who has been diagnosed with or is suspected of having multiple myeloma;

wherein the presence of expression of ikaros in the test sample indicates that the patient will respond to the treatment; and

wherein the absence of expression of ikaros in the test sample indicates that the individual will not respond to the treatment.

The invention provides a method for determining the likelihood that an individual will respond to a treatment for multiple myeloma, wherein the treatment comprises an immunomodulatory imide (IMid) compound and a hypomethylating agent, the method comprising:
determining the expression of cereblon in a test sample of exRNA from an individual who has been diagnosed with or is suspected of having multiple myeloma;

wherein a high level of expression of cereblon in the test sample indicates at the patient will not respond to the treatment, and

wherein a low level of expression of cereblon in the test sample indicates that the individual will respond to the treatment.

The present invention provides a method for determining the likelihood that an individual will respond to a treatment for multiple myeloma, wherein the treatment comprises an immunomodulatory imide (IMid) compound and a hypomethylating agent, the method comprising:
determining the expression of cereblon in a test sample of extracellular RNA (exRNA) from an individual who has previously received a treatment with an IMid;

wherein a high level of expression of cereblon in the test sample indicates at the patient will not respond to the treatment comprising an immunomodulatory imide (IMid) compound and a hypomethylating agent and

wherein a low level of expression of cereblon in the test sample indicates that the individual will respond to the treatment comprising an immunomodulatory imide (IMid) compound and a hypomethylating agent.

In one embodiment, the method further includes determining the expression one or more of ikaros and aiolos, and wherein when the individual has a low level of expression of cereblon, coupled with high levels of ikaros and/or aiolos prior to treatment, is indicative that the individual will likely respond to treatment. Conversely, where the individual has a high level of expression of cereblon, coupled with low levels of ikaros and/or aiolos prior to treatment, is indicative that the individual will likely not respond to treatment.

In one embodiment, the method further includes determining the expression of SPARC, and wherein the individual has a low level of expression of cereblon, coupled with a high level of SPARC prior to treatment, indicates that the individual will likely respond to the treatment. Conversely, where there is a high level of cereblon expression coupled with a low level of SPARC prior to treatment, this indicates that the individual will likely not respond to treatment.

Preferably, the individual (for whom a response to a treatment with an IMid and a hypomethylating agent is being determined) has previously received a treatment with an IMid.

The skilled person will appreciate that the terms “high level of expression” and “low level of expression” are intended as relative terms and are to be taken in the context of the specific patient group. In the present context, for example, a “high level of expression” may be taken to refer to a high copy number of gene exRNA transcripts in a given sample obtained from an individual having multiple myeloma, and compared to the average or typical levels of expression of the same gene in the overall cohort of multiple myeloma patients.

More specifically, as used herein, a “high level of expression of cereblon” may refer to copy numbers of the cereblon transcript in a sample of exRNA of at least 400, at least 450, preferably more than 470 copies/mL (preferably per mL of plasma).

As used herein, a “low level of expression of cereblon” may refer to copy numbers of the cereblon transcript in a sample of exRNA of less than 400, less than 300, or less than 100 copies/mL (preferably per mL of plasma).

As used herein, a “high level of expression of ikaros” may refer to copy numbers of the ikaros transcript in a sample of exRNA of at least 80, at least 100, preferably more than 120 copies/mL (preferably per mL of plasma).

As used herein, a “low level of expression of ikaros” may refer to copy numbers of the ikaros transcript in a sample of exRNA of less than 80, less than 50, or less than 20 copies/mL (preferably per mL of plasma).

As used herein, a “high level of expression of aiolos” may refer to copy numbers of the aiolos transcript in a sample of exRNA of at least 200, at least 240, preferably more than 250 copies/mL (preferably per mL of plasma).

As used herein, a “low level of expression of aiolos” may refer to copy numbers of the aiolos transcript in a sample of exRNA of less than 200, less than 100, or less than 50 copies/mL (preferably per mL of plasma).

The copy number will generally be considered to be “low” if it is lower than 20%, 30%, 40%, 50%, 60%, 70%, 80%, 90%, 100% or lower than the copy number observed in an individual having multiple myeloma but who has responded to a treatment with lenalidomide.

The copy number will generally be considered to be “high” if it is greater than 20%, 30%, 40%, 50%, 60%, 70%, 80%, 90%, 100% or higher than the copy number observed in an individual having multiple myeloma but who has not responded to a treatment with lenalidomide.

Preferably, the IMid is selected from lenalidomide, pomolidomide, thalidomide and apremilast.

In any embodiment, the treatment for multiple myeloma is selected from the group consisting of: azacitidine, lenalidomide, the combination of azacitidine and lenalidomide, or the combination of azacitidine, lenalidomide and dexamethasone.

The present invention also provides a kit for use in monitoring the response of an individual to a treatment for multiple myeloma, the kit comprising:

a means for detecting exRNA levels corresponding to one or more genes;

reagents for isolating or extracting exRNA from a peripheral blood sample of an individual.

Preferably, the kit also comprises written instructions for use of the kit in a method of the invention as described herein.

Preferably, the means for detecting exRNA levels from one or more genes is one or more nucleic acid probes or primers to either hybridize with a sequence from the one or more genes or amplify a sequence from the one or more genes. It is preferred that the probes are oligonucleotide probes, which bind to their target sites within the sequence of the one or more genes by way of complementary base-pairing. For the avoidance of doubt, in the context of the present invention, the definition of an oligonucleotide probe does not include the full length gene (or the complement thereof).

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Levels of baseline exRNA are biomarkers of prognosis

Random forest analysis of the top 5 exRNA at screening (baseline) (A) Best-fitting classification OS tree indicates that patients with low CRBN levels and high IKZF3 levels at screening are at low risk of progression (0.44) while patients with high CRBN and low SPARC levels have the highest risk of progression (3.3) (B) Kaplan-Meier plot for OS based on the groups identified by the classification tree (p=0.000003). (C) Kaplan-Meier plot for PFS based on a restricted classification tree consisting of CRBN, IKZF1 and IKZF3 indicating that high levels of CRBN at screening is a detrimental prognostic indicator (p=0.014) (D) Kaplan-Meier plot for OS based on a restricted classification tree consisting of CRBN, IKZF1 and IKZF3 indicating that patients with high levels of CRBN at screening are at a higher risk of progression (p=0.005).

FIG. 2: Alterations at C1D5 can be utilised as biomarkers of response to therapy

Random forest analysis of the top 5 exRNA with fold changes at C1D5 (A) Best-fitting classification PFS tree indicates that fold changes in IKZF1≥0.5 coupled with fold changes in IRF4<−0.07 were associated with low risk of PFS (0.49) and fold changes in IKZF1<0.05 was associated with high risk in PFS (2.1) (B) Kaplan-Meier plot for PFS based on the groups identified by the classification tree (p=0.0051) indicating that an increase in IKZF1 expression is a good prognostic biomarker of response to therapy (C) Best-fitting classification OS tree indicates that fold changes in IKZF1≥0.05 coupled with fold changes in TGFB1≥0.081 were associated with low risk of OS (0.42) and fold changes in IKZF1<0.05 was associated with high risk in OS (2.7) (D) Kaplan-Meier plot for OS based on the groups identified by the classification tree (p=0.0001) indicating that patients with an increase in IKZF1 at C1D5 have a lower risk of progression in OS. (E) Kaplan-Meier plot for PFS based on a restricted classification tree consisting of CRBN, IKZF1 and IKZF3 indicating that increased levels of IKZF1<0.05 at C1D5 indicates a higher risk of progression (PFS, p=0.0085) (F) Kaplan-Meier plot for PFS based on a restricted classification tree consisting of CRBN, IKZF1 and IKZF3 indicating that increased levels of IKZF1<0.05 at C1D5 indicates a higher risk of progression (OS) and that patients with increasing IKZF1 and CRBN are at low risk of progression (OS, p=0.0001). (G) A combination analysis of exRNA at screening and C1D5 was assessed with the tree-building restricted to CRBN, IKZF1 and IKZF3. Kaplan-Meier plot for PFS demonstrated that patients with low CRBN and high IKZF1 at screening did better than patients with high CRBN levels and low C1D5 CRBN levels (p=0.002). (H) Kaplan-Meier plot for OS based on restricted classification tree in a combination analysis of screening and C1D5 fold changes demonstrated that patients with low CRBN and high IKZF3 at screening did better than patients with high CRBN levels and low C1D5 CRBN levels (p=0.0001).

FIG. 3: Identification of biomarkers of poor prognosis in patients

Peripheral blood (PL) can address spatial heterogeneity in the multi-focal plasma cell malignancy, wherein BM can be only site specific. PL is a source of both cfDNA and exRNA, both of which can be used as biomarkers to predict response of a patient enrolled in a trial. Analyses of exRNA at screening and in response to treatment indicates that changes in exRNA corresponding to genes that are direct or indirect targets for the treatment for multiple myeloma, are an important predictor of response to treatment.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

Multiple Myeloma (MM) is a multi-focal genetically heterogeneous clonal plasma cell malignancy present at multiple intra-medullary sites within the bone marrow at diagnosis. During disease progression, the plasma cells evolve the capacity to grow independently of the BM milieu and thus proliferate outside of the BM, manifesting as extramedullary (EM) MM and/or plasma cell leukemia (PCL). The diagnosis and monitoring of MM relies on sequential bone marrow biopsies and the quantitation of biomarkers of disease burden in the blood and/or urine—clonal immunoglobulin (paraprotein, PP) and/or isotype restricted free-light chains (Serum Free light chains, SFLC or Bence Jones Proteinuria).

Mutational characterisation of multiple myeloma patients has conventionally utilised single-site bone marrow biopsies that are spatially and temporally-restricted. Thus, the diagnosis and monitoring of multiple myeloma, has typically relied on sequential bone marrow biopsies. It is now increasingly recognised that such an approach may fail to capture the spatial and temporal genetic heterogeneity of this multi-focal disease.

Assessment of response to therapy in MM has conventionally been through continuous monitoring of serum free light chains and/or paraprotein and multiple myeloma cell proportion in bone marrow biopsies. This approach is similarly limited in its capacity to provide information about the underlying tumour biology and specific response based on the presence of certain mutations or biomarkers.

In contrast to conventional approaches, the present invention provides a non-invasive approach for determining whether an individual is responding to a treatment for multiple myeloma. In particular, the methods of the present invention allow for early assessment of patient prognosis and response to treatment, within days of commencement of treatment.

The present invention therefore allows for more robust determination of patient prognosis and allows specific treatments to be aligned with the genetic alterations present in the disease. In particular, the methods of the present invention enable earlier intervention in circumstances where one treatment approach is no longer effective, facilitating the adaptation of the treatment protocol to minimise unnecessary exposure of the patient to treatments which are not efficacious but can have significant side effects.

The present inventors have found, through analysis of annotated sample sets from a phase 1b trial that it is possible to predict patient outcomes based on early alterations in exRNA levels. More specifically, the inventors have utilised quantitative analyses of exRNA of genes regulated by the treatment received by multiple myeloma patients, and determined that changes in exRNA levels immediately post-treatment can assist in determining response to the treatment.

The present inventors have therefore found that exRNA provides for an early indication of treatment success, such that it is possible to determine whether an individual is likely to benefit from a treatment within a matter of days of commencing that treatment. To date, no clinical trials in multiple myeloma patients have assessed the potential utility of exRNA to predict early response to therapy. Thus, the present invention provides a novel and significantly advantageous method for providing early feedback on responses of patients to treatment. The ability to obtain feedback on treatment response so early in the treatment protocol provides invaluable advice to clinicians, but also invaluable time to patients such that patients who are not responding can be transferred to an alternative treatment plan without having to undergo further treatment.

The current invention thereby also provides the clinician or physician caring for a subject with multiple myeloma with information about the likelihood of response to treatment and overall survival. On the basis of the results of the method of the invention, the clinician or physician can:
(i) avoid treating a subject with a treatment regime that the subject is unlikely to respond to;
(ii) avoid treating a subject with a treatment regime that will provide side effects and unlikely to provide any benefit in treating the disease;
(iii) enroll the patient in clinical trials for new therapies for multiple myeloma,
(iv) treat the subject with alternative therapies, such as those which target an alternative oncogenic signalling pathway;
(v) discuss the likely treatment and outcome scenarios with the subject;
(vi) provide more regular or extensive post-treatment surveillance for a subject identified as having a low response or survival rate; and/or
(vii) proceed to treat a subject identified as likely to response with added confidence the treatment is likely to provide benefit to the subject.

Biomarkers

The skilled person will be familiar with methods for determining expression levels, including changes therein, including changes in copy numbers for the following biomarkers:

As used herein, “cereblon” (also referred to as CRBN, MRT2 or MRT2A) refers to a 442-amino acid protein conserved from plant to human. At least two isoforms of the protein cereblon (CRBN) exist, which are 442 and 441 amino acids long, respectively, The UniProt accession code for the human protein is Q96SW2. In humans, CRBN was initially characterized as an RGS-containing novel protein that interacted with a calcium-activated potassium channel protein (SLO 1) in the rat brain, and was later shown to interact with a voltage-gated chloride channel (CIC-2) in the retina with AMPK7 and DDBI. (See Jo et al., J Neurochem, 2005, 94: 1212-1224). CRBN has also been identified as a target for the development of therapeutic agents for diseases of the cerebral cortex. (See WO 2010/137547). In any embodiment of the present invention, the form of CRBN identified includes an isoform of CRBN.

As used herein, ikaros (IKZF1) refers to “DNA-binding protein Ikaros” also known as “Ikaros family zinc finger protein 1”. The protein in humans is encoded by the IKZF1 gene. Ikaros displays crucial functions in the hematopoietic system and its loss of function has been linked to the development of lymphoid leukemia. In particular, Ikaros has been found in recent years to be a major tumor suppressor involved in human B-cell acute lymphoblastic leukemia. IKZF1 is upregulated in granulocytes, B cells, CD4 and CD8 T cells, and NK cells, and downregulated in erythroblasts, megakaryocytes and monocytes. In Ikaros knockout mice, T cells but not B cells are generated late in mouse development due to late compensatory expression of the related gene Aiolos (IKZF3). Ikaros point mutant mice are embryonic lethal due to anemia; they have severe defects in terminal erythrocyte and granulocyte differentiation, and excessive macrophage formation. Several alternatively spliced transcript variants encoding different isoforms have been described for this gene. All isoforms share a common C-terminal domain, which contains two zinc finger motifs that are required for hetero- or homo-dimerization and for interactions with other proteins. The isoforms, however, differ in the number of N-terminal zinc finger motifs that bind DNA and contain the nuclear localization signal, resulting in members with and without DNA-binding properties. Only few isoforms contain the requisite three or more N terminal zinc motifs that confer high affinity binding to a specific core DNA sequence element in the promoters of target genes. The non-DNA-binding isoforms are largely found in the cytoplasm, and thought to function as dominant negative factors.

As used herein, aiolos (IKZF3 or ZNFN1A3) refers to “Zinc finger protein Aiolos”, also known as Ikaros family zinc finger protein 3 is a protein that in humans is encoded by the IKZF3 gene. This gene encodes a member of the Ikaros family of zinc-finger proteins. Three members of this protein family (Ikaros, Aiolos and Helios) are hematopoietic-specific transcription factors involved in the regulation of lymphocyte development. This gene product is a transcription factor that is important in the regulation of B lymphocyte proliferation and differentiation. Both Ikaros and Aiolos can participate in chromatin remodeling. Regulation of gene expression in B lymphocytes by Aiolos is complex as it appears to require the sequential formation of Ikaros homodimers, lkaros/Aiolos heterodimers, and Aiolos homodimers. At least six alternative transcripts encoding different isoforms have been described.

As used herein, IRF4 refers to Interferon regulatory factor 4 also known as MUM1, a protein that in humans is encoded by the IRF4 gene. Other synonyms include LSIRF, NF-EMS, and SHEP8. IRF4 is a transcription factor that has been implicated in acute leukemia. This gene is strongly associated with pigmentation: sensitivity of skin to sun exposure, freckles, blue eyes, and brown hair color.

As used herein, TGFβ1 refers to Transforming growth factor beta 1, a polypeptide member of the transforming growth factor beta superfamily of cytokines. Also known as CED, DPD1, LAP, TGFB, TGFbeta, TGFβ1 is a secreted protein that performs many cellular functions, including the control of cell growth, cell proliferation, cell differentiation, and apoptosis. In humans, TGFβ1 is encoded by the TGFB1 gene. TGF-β is a multifunctional set of peptides that controls proliferation, differentiation, and other functions in many cell types. TGF-β acts synergistically with TGFA in inducing transformation. It also acts as a negative autocrine growth factor. Dysregulation of TGF-β activation and signaling may result in apoptosis. Many cells synthesize TGF-β and almost all of them have specific receptors for this peptide. TGF-β1, TGF-β2, and TGF-β3 all function through the same receptor signaling systems.

As used herein, SPARC refers to Osteonectin (ON), also known as secreted protein acidic and rich in cysteine (SPARC) or basement-membrane protein 40 (BM-40). It is a protein that in humans is encoded by the SPARC gene. Osteonectin is a glycoprotein in the bone that binds calcium. It is secreted by osteoblasts during bone formation, initiating mineralization and promoting mineral crystal formation. Osteonectin also shows affinity for collagen in addition to bone mineral calcium. A correlation between osteonectin over-expression and ampullary cancers and chronic pancreatitis has been found.

exRNA

Extracellular RNA (also known as exRNA or exosomal RNA) describes RNA species present outside of the cells from which they were transcribed. exRNA may be found in bodily fluids such as venous blood, saliva, breast milk, urine, semen, menstrual blood, and vaginal fluid. “Extracellular RNA” defines a group of several types of RNAs whose functions are diverse, yet they share a common attribute of existence in an extracellular environment. exRNA may include the following types of RNA: messenger RNA (mRNA), transfer RNA (tRNA), microRNA (miRNA), small interfering RNA (siRNA) and long non-coding RNA (IncRNA).

Although not completely understood, the population of exRNA found in a biological sample is thought to be comprised of exRNA from both healthy and unhealthy (e.g., tumour) cells. Thus, observing alterations in the exRNA profile for a given gene provides a snap-shot of the whole-body response (the contribution of healthy and tumour cells) to the treatment received.

A ‘cell-free nucleic acid’, or “exRNA” as used herein, is a nucleic acid, preferably RNA (genomic or mitochondrial), that has been released or otherwise escaped from a cell into blood or other body fluid in which the cell resides. The extraction or isolation of cell-free nucleic acid (e.g. RNA) from a body fluid, such as peripheral blood, does not involve the rupture of any cells present in the body fluid. Cell-free RNA may be RNA isolated from a body fluid in which all or substantially all particulate material in the fluid, such as cells or cell debris, has been removed.

Cell-free nucleic acids, such as RNA (exRNA), may be extracted from peripheral blood samples using techniques including e.g. Lo et al, U.S. Pat. No. 6,258,540; Huang et al, Methods Mol. Biol, 444: 203-208 (2008); and the like, which are incorporated herein by reference. By way of non-limiting example, peripheral blood may be collected in EDTA or Streck BCT RNA tubes, after which it may be fractionated into plasma, white blood cell, and red blood cell components by centrifugation. DNA present in the cell-free plasma fraction (e.g. from 0.5 to 2.0 mL) may be extracted using a QIAamp DNA Blood Mini Kit (Qiagen, Valencia, Calif.), QIAamp circulating nucleic acid kit (Qiagen, Hilden, Germany), or like kit, in accordance with the manufacturer's protocol. exRNA samples are preferably treated to remove any contaminating genomic DNA, for example using a Turbo DNA-free kit (Thermo Fisher Scientific MA, USA) or like kit, according to manufacturer recommendations.

As used herein, the term ‘nucleic acid’ refers to any molecule, preferably a polymeric molecule, incorporating units of ribonucleic acid or an analog thereof. The nucleic acid can be either single-stranded or double-stranded.

The term ‘isolated’ or ‘partially purified’ as used herein refers, in the case of a nucleic acid, to a nucleic acid separated from at least one other component (e.g., nucleic acid or polypeptide) that is present with the nucleic acid as found in its natural source and/or that would be present with the nucleic acid when expressed by a cell. A chemically synthesized nucleic acid or one synthesized using in vitro transcription/translation is considered ‘isolated’.

As used herein, a ‘portion’ of a nucleic acid molecule refers to contiguous set of nucleotides comprised by that molecule. A portion can comprise all or only a subset of the nucleotides comprised by the molecule. A portion can be double-stranded or single-stranded.

As used herein, ‘amplified product’, ‘amplification product’, or ‘amplicon’ refers to oligonucleotides resulting from an amplification reaction that are copies of a portion of a particular target nucleic acid template strand and/or its complementary sequence, which correspond in nucleotide sequence to the template nucleic acid sequence and/or its complementary sequence. An amplification product can further comprise sequence specific to the primers and which flanks sequence which is a portion of the target nucleic acid and/or its complement. An amplified product, as described herein will generally be double-stranded DNA, although reference can be made to individual strands thereof.

In any method of the invention described herein, assessing or determining in a sample of an amount, level, presence of, (a) circulating cell-free tumor-derived nucleic acid or circulating tumour free nucleic acids, or (b) cell-free nucleic acids, or (c) exRNA may be by any method as described herein, for example a form of PCR, microarray, sequencing etc.

An amount of a nucleic acid may be quantified using any method described herein, or for example, the polymerase chain reaction (PCR) or, specifically quantitative polymerase chain reaction (qPCR) or droplet digital polymerase chain reaction (ddPCR). QPCR is a technique based on the polymerase chain reaction, and is used to amplify and simultaneously quantify a targeted nucleic acid molecule. QPCR allows for both detection and quantification (as absolute number of copies or relative amount when normalized to DNA input or additional normalizing genes) of a specific sequence in a DNA sample. The procedure follows the general principle of polymerase chain reaction, with the additional feature that the amplified DNA is quantified as it accumulates in the reaction in real time after each amplification cycle. QPCR is described, for example, in Kurnit et al. (U.S. Pat. No. 6,033,854), Wang et al. (U.S. Pat. Nos. 5,567,583 and 5,348,853), Ma et al. (The Journal of American Science, 2(3), 2006), Heid et al. (Genome Research 986-994, 1996), Sambrook and Russell (Quantitative PCR, Cold Spring Harbor Protocols, 2006), and Higuchi (U.S. Pat. Nos. 6,171,785 and 5,994,056). The contents of these are incorporated by reference herein in their entirety.

Preferably, the amount of a nucleic acid sample is determined using droplet digital PCT technology, which can incorporate absolute quantification without the need for a reference sample.

As used herein, the Tm is the temperature (under defined ionic strength and pH) at which 50% of the target sequence binds to a perfectly matched probe. In this regard, the Tm of probes of the present invention, at a salt concentration of about 0.02M or less at pH 7, is preferably above 40° C. and preferably below 70° C., more preferably about 53° C. Premixed binding solutions are available (eg. EXPRESSHYB Hybridisation Solution from CLONTECH Laboratories, Inc.), and binding can be performed according to the manufacturers instructions. Alternatively, one of a skill in the art can devise variations of these binding conditions.

Following binding, washing under stringent (preferably highly stringent) conditions removes unbound nucleic acid molecules. Typical stringent washing conditions include washing in a solution of 0.5-2× SSC with 0.1% SDS at 55-65° C. Typical highly stringent washing conditions include washing in a solution of 0.1-0.2× SSC with 0.1% SDS at 55-65° C. A skilled person can readily devise equivalent conditions for example, by substituting SSPE for the SSC in the wash solution.

Apart from the stringency of the hybridization conditions, hybridization specificities may be affected by a variety of probe design factors, including the overall sequence similarity, the distribution and positions of mismatching bases, and the amount of free energy of the RNA duplexes formed by the probe and target sequences.

The ‘complement’ of a nucleic acid sequence binds via complementary basepairing to said nucleic acid sequence. A non-coding (anti-sense) nucleic acid strand is also known as a “complementary strand”, because it binds via complementary base-pairing to a coding (sense) strand.

In one aspect, the probe may be immobilised onto a support or platform. Immobilising the probe provides a physical location for the probe, and may serve to fix the probe at a desired location and/or facilitate recovery or separation of probe.

The support may be a rigid solid support made from, for example, glass or plastic, or else the support may be a membrane, such as nylon or nitrocellulose membrane. 3D matrices are suitable supports for use with the present invention—eg. polyacrylamide or PEG gels.

In one embodiment, the support may be in the form of one or more beads or microspheres, for example in the form of a liquid bead microarray. Suitable beads or microspheres are available commercially (eg. Luminex Corp., Austin, Tex.). The surfaces of the beads may be carboxylated for attachment of RNA. The beads or microspheres may be uniquely identified, thereby enabling sorting according to their unique features (for example, by bead size or colour, or a unique label), In one aspect, the beads/microspheres are internally dyed with fluorophores (eg. red and/or infrared fluorophores) and can be distinguished from each other by virtue of their different fluorescent intensity.

In one aspect, prior to contacting the nucleotide sequence of a gene with said oligonucleotide probe, the method further comprises the step of amplifying a portion of the gene, or the complement thereof, thereby generating an amplicon.

It may be desirable to amplify the target nucleic acid if the sample is small and/or comprises a heterogeneous collection of RNA sequences.

Amplification may be carried out by methods known in the art, and is preferably carried out by ddPCR. A skilled person would be able to determine suitable conditions for promoting amplification of a nucleic acid sequence.

Thus, in one aspect, amplification is carried out using a pair of sequence specific primers, wherein said primers bind to target sites in the gene, or the complement thereof, by complementary basepairing. In the presence of a suitable DNA polymerase and DNA precursors (dATP, dCTP, dGTP and dTTP), the primers are extended, thereby initiating the synthesis of new nucleic acid strands which are complementary to the individual strands of the target nucleic acid. The primers thereby drive amplification of a portion of the gene, or the complement thereof, thereby generating an amplicon. This amplicon comprises the target sequence to which the probe binds, or may be directly sequenced to identified the presence of one or more mutations as described herein.

For the avoidance of doubt, in the context of the present invention, the definition of an oligonucleotide primer does not include the full length gene (or complement thereof).

The primer pair comprises forward and reverse oligonucleotide primers. A forward primer is one that binds to the complementary, non-coding (antisense) strand of the target nucleic acid and a reverse primer is one that binds to the corresponding coding (sense) strand of the target nucleic acid.

Primers are designed to bind to the target gene sequence based on the selection of desired parameters, using conventional software, such as Primer Express (Applied Biosystems). In this regard, it is preferred that the binding conditions are such that a high level of specificity is provided. The melting temperature (Tm) of the primers is preferably in excess of 50° C. and is most preferably about 60° C. A primer of the present invention preferably binds to target nucleic acid but is preferably screened to minimise self-complementarity and dimer formation (primer-to-primer binding).

The forward and reverse oligonucleotide primers are typically 1 to 40 nucleotides long. It is an advantage to use shorter primers, as this enables faster annealing to target nucleic acid.

Preferably the forward primer is at least 10 nucleotides long, more preferably at least 15 nucleotides long, more preferably at least 18 nucleotides long, most preferably at least 20 nucleotides long, and the forward primer is preferably up to 35 nucleotides long, more preferably up to 30 nucleotides long, more preferably up to 28 nucleotides long, most preferably up to 25 nucleotides long. In one embodiment, the forward primer is about 20-21 nucleotides long.

Preferably the reverse primers are at least 10 nucleotides long, more preferably at least 15 nucleotides long, more preferably at least 20 nucleotides long, most preferably at least 25 nucleotides long, and the reverse primers are preferably up to 35 nucleotides long, more preferably up to 30 nucleotides long, most preferably up to 28 nucleotides long. In one embodiment, the reverse primer is about 26 nucleotides long.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nl, to a few hundred pl, e.g. 200 μl. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time FOR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences.

Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, p2-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Droplet digital PCR” (ddPCR) refers to a digital PCR assay that measures absolute quantities by counting nucleic acid molecules encapsulated in discrete, volumetrically defined, water-in-oil droplet partitions that support PCR amplification (Hindson et al (2011) Anal Chem 83:8604-8610, the entire contents of which are hereby incorporated by reference). A single ddPCR reaction may be comprised of at least 20,000 partitioned droplets per well.

A “droplet” or “water-in-oil droplet” refers to an individual partition of the droplet digital PCR assay. A droplet supports PCR amplification of template molecule(s) using homogenous assay chemistries and workflows similar to those widely used for real-time PCR applications (Hinson et al., 2011, Anal. Chem. 83:8604-8610; Pinheiro et al., 2012, Anal. Chem. 84:1003-1011).

Droplet digital PCR may be performed using any platform that performs a digital PCR assay that measures absolute quantities by counting nucleic acid molecules encapsulated in discrete, volumetrically defined, water-in-oil droplet partitions that support PCR amplification. The strategy for droplet digital PCR may be summarized as follows: a sample is diluted and partitioned into thousands to millions of separate reaction chambers (water-in-oil droplets) so that each contains one or no copies of the nucleic acid molecule of interest. The number of “positive” droplets detected, which contain the target amplicon (i.e., nucleic acid molecule of interest), versus the number of “negative” droplets, which do not contain the target amplicon (i.e., nucleic acid molecule of interest), may be used to determine the number of copies of the nucleic acid molecule of interest that were in the original sample. Examples of droplet digital PCR systems include the QX100™ Droplet Digital PCR System by Bio-Rad, which partitions samples containing nucleic acid template into 20,000 nanoliter-sized droplets; and the RainDrop™ digital PCR system by RainDance, which partitions samples containing nucleic acid template into 1,000,000 to 10,000,000 picoliter-sized droplets.

Digital droplet PCR (ddPCR) takes advantage of recent developments in microfluids and surfactant chemistries. The reaction mixture is divided into approximately 20000 droplets which are PCR amplified, post-FOR fluorescently labeled and read in an automated droplet flow cytometer. Each droplet is assigned a positive and negative (1 or 0) value based on their fluorescent intensity. The amount of positives and negatives are read D by flow cytometer and are used to calculate the concentration and the 95% Poisson confidence levels. The fundamental advantages that digital droplet PCR (ddPCR) offers are many, including (a) an increase in dynamic range, (b) improvement in precision of detecting small changes in template DNA, (c) its ability to tolerate a wide range of amplification efficiencies, and (d) its ability to measure absolute DNA/RNA concentrations.

The skilled person will be familiar with methods for determining fold change in exRNA levels after quantification of exRNA levels using any method described herein, or familiar to those skilled in the art.

In any method of the invention, determining whether a treatment has been successful, or the likelihood of a positive prognosis for an individual can be determined by measuring the fold change in exRNA levels, or a change in copy numbers for one or more of cereblon, ikaros, aiolos, IRF4 or other gene that is regulated by the treatment received by the individual.

As used herein ‘reference score’, ‘cut-off value’, ‘survival cut-off’ or ‘tree cut-off’ are used interchangeably. Preferably, the reference score has been predetermined or is determined from a cohort of patients with known multiple myeloma outcome, preferably survival (e.g. overall survival, 1 year, 2 years, 3 years or 4 years survival) after treatment with a regime for multiple myeloma. The reference score stratifies the subject into one of two subgroups with the following rule: where there is an increase in exRNA levels for a gene that is positively regulated by the treatment, or a decrease in exRNA levels for a gene that is negatively regulated by the treatment, then the patient is assigned to the group with a high likelihood of response (e.g., high likelihood of a positive prognosis, high likelihood of progression free survival or of overall survival). On the other hand, where there is an increase or no change in exRNA levels for a gene that is negatively regulated by the treatment, or a decrease or no change in exRNA levels for a gene that is positively regulated by the treatment, then the patient is assigned to the group with low likelihood of response (e.g. low likelihood of progression free survival, or of overall survival).

Preferably, the reference score stratifies subjects into a group with a high likelihood of overall survival of at least 95%, 94%, 93%, 92%, 91%, 90%, 85%, 80%, 75% or 70%, and into a group with a low likelihood of overall survival of less than 60%, 55%, 54%, 53%, 52%, 51%, 50%, 49%, 48%, 47%, 46%,45% or 44%. The reference score can be determined using statistical methods known in the art, for example, a tree-structure recursive partitioning statistical model or the median value. Survival analysis may be performed using Kaplan-Meier method and the two survival curves from the two subgroups can be compared using the log-rank test, such as that described in the Examples.

The Kaplan-Meier method (also known as the product limit estimator) estimates the survival function from life-time data. In medical research, it can be used to measure the fraction of patients living for a certain amount of time after treatment.

A plot of the Kaplan-Meier method of the survival function is a series of horizontal steps of declining magnitude which, when a large enough sample is taken, approaches the true survival function for that population. The value of the survival function between successive distinct sampled observations (“clicks”) is assumed to be constant.

An important advantage of the Kaplan-Meier curve is that the method can take into account “censored” data—losses from the sample before the final outcome is observed (for instance, if a patient withdraws from a study). On the plot, small vertical tick-marks indicate losses, where patient data has been censored. When no truncation or censoring occurs, the Kaplan-Meier curve is equivalent to the empirical distribution.

Survival analysis can be performed using the Kaplan-Meier method (as described in the Examples herein).

Methods for Monitoring Disease Progression and Treatment Efficacy

The present invention can be used to diagnose, monitor disease progression or treatment efficacy in an individual.

Monitoring disease progression or treatment efficacy may be of an individual having any type of multiple myeloma including smouldering or indolent multiple myeloma, active multiple myeloma, multiple solitary plasmacytomas, extramedullary plasmacytoma, secretory, non-secretory, IgG lambda or kappa light chain (LC) types. The most common immunoglobulins (Ig) made by myeloma cells in multiple myeloma are IgG, IgA and IgM, less commonly, IgD or IgE is involved.

Aspects of the present invention, such as monitoring disease progression or treatment efficacy, may be particularly useful in individuals where no conventional peripheral blood biomarker (e.g. no paraprotein, or other marker described herein including the Examples, or known in the art) is detectable.

The methods of the present invention typically include a comparison of exRNAs from the individual (sometimes referred to as a “test sample”) with exRNA in a control profile.

In some instances, the ‘control profile’ may include the level of exRNA from a peripheral blood sample of an individual or individuals that do not have any clinically or biochemically detectable multiple myeloma. In such instances, the peripheral blood sample of an individual or individuals that do not have any clinically or biochemically detectable multiple myeloma is herein referred to as the ‘control sample’. The ‘control profile’ may be derived from an individual that, but for an absence of multiple myeloma, is generally the same or very similar to the individual selected for determination of whether they have multiple myeloma. The measurement of the exRNA corresponding to a particular gene in the control sample from the peripheral blood of the individual or individuals for deriving the control profile is generally done using the same assay format that is used for measurement of the exRNA in the test sample.

It will be appreciated that the control profile may also be derived from the same individual from which the test sample is taken, but at a different time-point, for example, a year or several years earlier. As such, the control profile may also include the level of exRNA from the individual before the individual received treatment for multiple myeloma, or at an earlier stage during the treatment of multiple myeloma. Such a control profile thereby forms a baseline or basal level profile of the level of exRNA in the individual, against which the test sample may be compared.

A control profile for measuring disease progression or monitoring treatment efficacy may be generated from the same individual from which the test sample is taken, but at a different time-point, for example, a year or several years earlier. Such a control profile thereby forms a baseline or basal level profile in the individual of the level of exRNA, in particular corresponding to exRNA from the genes regulated by the treatment received by the individual.

In the present specification failure of treatment (or where the individual is considered not to have responded to treatment) includes progression of disease while receiving a treatment (e.g. chemotherapy) regimen without experiencing any transient improvement, no objective response after receiving one or more cycles of a treatment regimen or a limited response with subsequent progression while receiving a treatment regimen. Myeloma that is not responsive to therapy may also be termed ‘Refractory multiple myeloma’. Refractory myeloma may occur in patients who never see a response from their treatment therapies or it may occur in patients who do initially respond to treatment, but do not respond to treatment after relapse.

In the present specification ‘relapse’ means, unless otherwise specified, the return of signs and symptoms of cancer after a period of improvement.

In the present specification, success of treatment (or where the individual is considered to have responded to treatment), includes stabilisation of the disease or the slowing down or cessation of disease progression. ‘Response to treatment’ refers to therapeutic treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment can also mean prolonging survival as compared to expected survival if not receiving treatment. Treatment may not necessarily result in the complete clearance of a disease or disorder but may reduce or minimise complications and side effects of infection and the progression of a disease or disorder.

As used herein, a positive response of an individual to a treatment includes an increase in the progression free survival of the individual. Alternatively, a positive response of an individual to a treatment includes an increase in the overall survival of the individual. Accordingly, the present invention also finds utility in predicting the overall survival or progression free survival of an individual receiving/requiring treatment for multiple myeloma.

As used herein “overall survival” (OS) refers to the length of time from either the date of diagnosis or the start of treatment for a disease, such as cancer, that patients diagnosed with the disease are still alive. In a clinical trial, measuring the overall survival is one way to see how well a new treatment works.

‘Overall survival’ or ‘OS’ is well known to one of skill in the art and refers to the fate of the patient after an event, preferably after the start or end of a treatment regime, despite the possibility that the cause of death in a patient is not directly due to the effects of the disease (cancer). In other words, it refers to the prognosis that the patient will not die because of multiple myeloma, preferably within at least 1 year, at least 2 years, at least 3 years, at least 4 years, at least 5 years, at least 10 years or at least 15 years.

As used herein “progression free survival” (PFS) refers to the length of time during and after the treatment of a disease, such as cancer, that a patient lives with the disease but it does not get worse. In a clinical trial, measuring the progression-free survival is one way to see how well a new treatment works.

‘Prognosis’ generally refers to a forecast or prediction of the probable course or outcome of the multiple myeloma. As used herein, prognosis includes the forecast or prediction of any one or more of the following: duration of survival of a patient susceptible to or diagnosed with multiple myeloma, duration of recurrence-free survival, duration of progression free survival of a patient susceptible to or diagnosed with multiple myeloma, response rate in a group of patients susceptible to or diagnosed with multiple myeloma, and/or duration of response in a patient or a group of patients susceptible to or diagnosed with a multiple myeloma. Prognosis also includes prediction of favorable responses to multiple myeloma treatments, such as a conventional multiple myeloma therapy, for example a treatment regime including a hypomethylating agent (such as azacytine) and an IMid (such as lenolinamide), and combinations thereof. As will be understood by those skilled in the art, the prediction may not need to be correct for 100% of the subjects evaluated. The term, however, requires that a statistically significant portion of subjects can be identified as having an increased probability of having a given outcome.

‘Responding to a treatment regime’ refers to a clinically or biochemically favorable detectable response to a treatment, such as a conventional multiple myeloma therapy. Typically, a favorable response is survival measured at a later time point after treatment, for example, 1, 2, 3, or 4 years post treatment.

Although the invention finds application in humans, the invention is also useful for therapeutic veterinary purposes. The invention is useful for domestic or farm animals such as cattle, sheep, horses and poultry; for companion animals such as cats and dogs; and for zoo animals.

The present invention includes monitoring the efficacy of a treatment for multiple myeloma, wherein the treatment includes but is not limited to administration of any one or more of: Dexamethasone, Cyclophosphamide, Thalidomide, Lenalinomide, Etopside, Cisplatin, Ixazomib, Bortezomib, Vemurafinib, Rigosertib, Trametinib, Panobinostat, Azacytidine, Pembrolizumab, Nivolumumab, Durvalumab or autologous stem cell transplant (ASCT).

The treatment may include one or more drugs, or any combination of two or more drugs including in the following combinations: Dexamethasone, Cyclophosphamide, Etoposide and Cisplatin (DCEP); Dexamethasone, Cyclophosphamide, Etoposide, Cisplatin and Thalidomide (T-DCEP); Azacytidine and Lenalidomide (Rd), Ixazomib-cyclophosphamide-dexamethasone (ICd), or Bortezomib, Cyclophosphamide and Dexamethasone (VCD). The treatment may include combinations of DCEP, T-DCEP, Rd, lcd or VCD in combination with additional drugs.

The present invention also includes adapting or modifying a treatment for multiple myeloma based on the results of determining or monitoring the mutational status of an individual receiving treatment for multiple myeloma. The adaption or modification may include removing a particular drug or drugs from the treatment protocol and replacing the drug with one or more alternative drugs. Alternatively, the adaptation or modification may include supplementing the existing treatment with additional drugs.

In any embodiment, the replacement or supplemental treatment includes administering any one or more of Dexamethasone, Cyclophosphamide, Thalidomide, Lenalinomide, Pomalidomide, Etoposide, Cisplatin, Bortezomib, Carfilzomib, Cobimetinib, Ixazomib, Selumetinib, Trametinib, Vemurafinib, Panobinostat, Vorinostat, Azacytidine, Venetoclax, Daratumumab, Pembrolizumab, Nivolumumab, Durvalumab or autologous stem cell transplant (ASCT). The replacement or supplemental treatment may also include administering any one or more of the combinations of: Dexamethasone, Cyclophosphamide, Etoposide and Cisplatin (DCEP); Dexamethasone, Cyclophosphamide, Etoposide, Cisplatin and Thalidomide (T-DCEP); Lenalidomide and Dexamethanasone (Rd), Ixazomib-cyclophosphamide-dexamethasone (ICd); or Bortezomib, Cyclophosphamide and Dexamethasone (VCD). The treatment may include combinations of DCEP, T-DCEP, Rd, lcd or VCD in combination with additional drugs.

### EXAMPLES

Methods:

Uniformly Treated MM Patients

A phase 1b, single-centre study of oral AZA, a hypomethylating agent, in combination with RD for the treatment of R/R MM patients was approved by the Alfred Hospital Human Ethics Committee and provided a platform in which to access a population of uniformly treated MM patients for the purposes of conducting a correlative study. A total of 24 heavily pre-treated R/R MM patients were enrolled. LEN (25 mg) was given on days 1-21 of a 28-day cycle, and DEX (40 mg) was given on days 1, 8, 15 and 22. AZA was given in escalating doses, with an initial dose of 100 mg for days 1 to 14, which then increased by either 7 days or 50 mg/cohort, not exceeding a maximum dose of 200 mg from 1 to 21 days per 28-day cycle. Treatment continued until progression or toxicity occurred, or patient consent was withdrawn. Objective response rate (ORR) as per the IMWG uniform response rate criteria was used to categorise patients as responders (partial response (PR), very good partial response (VGPR) or complete response (CR)) or non-responders (minimal response (MR), stable disease (SD) and progressive disease (PD)). Progression-free survival (PFS) was measured from the date of commencing therapy to the date of relapse/progression or death from any cause, whichever occurred first. Overall survival (OS) was measured from the date of first commencing therapy.

Correlative Study—Peripheral Blood (PB) Collection and Processing

Peripheral blood PL in Streck BCT DNA and RNA tubes (Streck, NE, USA) were collected at screening, cycle 1 day 5 (C1D5), C1D15, end of cycle 3 (EOC3) and EOC6 following informed consent or the purposes of the correlative study. Further PL samples were collected in patients who responded past this timeframe. Immediately upon sample collection, the tubes were inverted to mix the blood with the preservative in the collection tube. PL was separated from PB through centrifugation at 820 g for 10 minutes (mins) within 24 hours of sample collection. Supernatant was collected without disturbing the cellular layer and centrifuged again at 16,000 g for 10 mins to remove any residual cellular debris and stored at −800 C. in 1 ml aliquots for long-term storage until isolation.

Cell-free RNA Extraction

Frozen PL samples were used for exRNA extraction using the QIAamp circulating nucleic acid kit (Qiagen, Hilden, Germany) according to manufacturers' instructions. Approximately, 3 mls of PL was used for extractions. Identical procedure was followed for exRNA, until elution stage. exRNA was treated for genomic DNA contamination using the Turbo DNA-free kit, according to manufacturers' recommendations (Thermo Fisher Scientifc, Mass., USA). Subsequently, PL exRNA was quantified with a QUBIT Fluorometer 3.0 and high sensitivity DNA and RNA detection kits (Thermo Fisher Scientific). The maximum input volume utilised for the QUBIT assay was 5 μl. The extracted exRNA were stored at −80° C. until further processing.

Isolation of Mononuclear Cells and MM Cells

Peripheral blood mononuclear cells (PBMC) in EDTA tubes and BM aspirates were collected at screening, C1D5, EOC3 (or C4D1), EOC6 (C7D1), and at relapse or progression. BM aspirates at screening from MM patients and PB from EDTA tubes were subjected to ficoll isolation of bone marrow mononuclear cells (BMMNC) and PBMC, respectively, as previously described (Mithraprabhu et al. 2014 Epigenetics. 9(11):1511-1520). PBMC were snap frozen as cell pellets and stored at −800 C. until further analysis. BMMNC were assessed for determination of MM cell proportions through flow cytometry and subsequent isolation using CD138+ magnetic beads (Miltenyi, Bergisch Gladbach, Germany; (Mithraprabhu et al. 201, supra). Samples were snap frozen and stored as before. Of the 24 patients enrolled for the trial, 15 patients had sufficient CD138+ for further analysis.

Assessment of exRNA Gene Expression

For cfRNA assessment, 12 genes were selected based on previously published literature. The one-step ddPCR supermix kit (Biorad) was used for quantitative assessment utilising 2 μL of cfRNA elution for each well with samples run as duplicates. All primers used for cfRNA tracking were obtained from Biorad and as described in Mithraprabhu et al (2019) Leukemia 33: 2022-2033. A minimum of three positive droplets between the two wells was required for a positive result. Quantasoft™ software version 1.7 enabled the determination of the number of copies in the reaction. Absolute number of copies in 1 ml of PL was calculated as follows: if there were A copies/μl and a total of B μl of the PCR mix was made, then a total of A×B=AB copies of the gene is present in the PCR mix. Since, 2 μl of the sample was added into the reaction mix, AB/2=C copies/μl of gene was present in the starting material. Therefore, if 3 ml of PL sample was used for extraction and cfRNA was eluted in 50 μl volume, 50×C=D copies are present in total. Finally, D/3=E copies of gene is present in 1 ml PB PL in this patient. Subsequently, from this value, a fold-change from screening to C1D5 was calculated to assess whether there was an initial change in the expression of these genes with treatment.

Patients were categorised as “exRNA increased” if there was an increase in the number of copies/ml of PL in the C1D5 compared to screening and “exRNA decreased” if screening had more copies. Patients that did not express genes at both time points were excluded from the survival analyses. For gene expression in BM or PBMC, four reference genes, ACTB, RPL30, HPRT1 and GAPDH were analysed in addition to selected targets. An average concertation of these four reference genes was utilised to derive normalised expression levels of target genes in BM and PBMC at the specific timepoints analysed.

Statistical Analyses

The random survival forests methodology (as implemented in the R package randomForestSRC 2.4.1) was used to identify the exRNAs most closely associated with PFS and OS. The levels of exRNA at screening and fold changes in exRNA at C1D5 compared to screening were determined.

All statistical analyses were performed using Graph Pad Prism 7.0f.

Results:

High CRBN exRNA at Screening is Associated with a High Risk of Progression

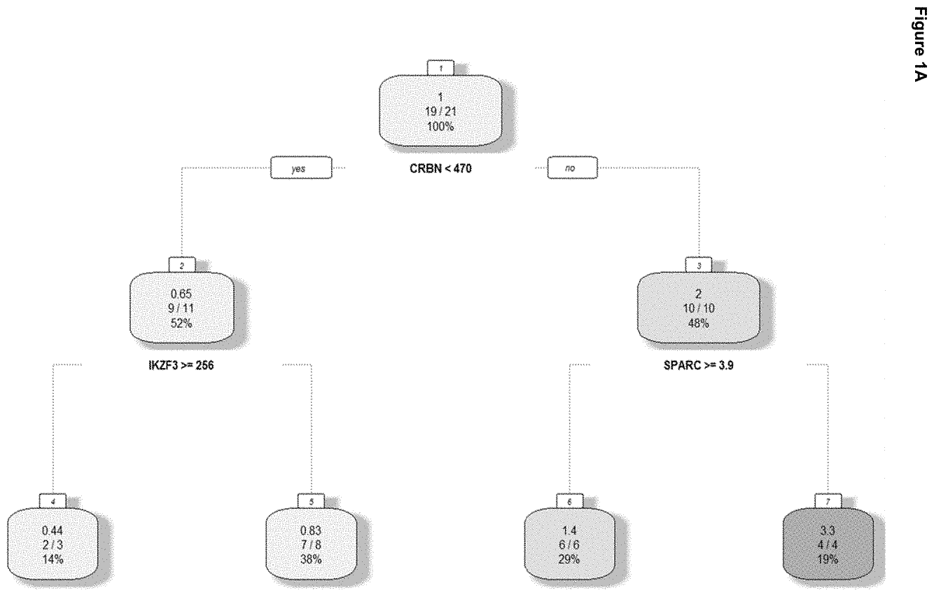

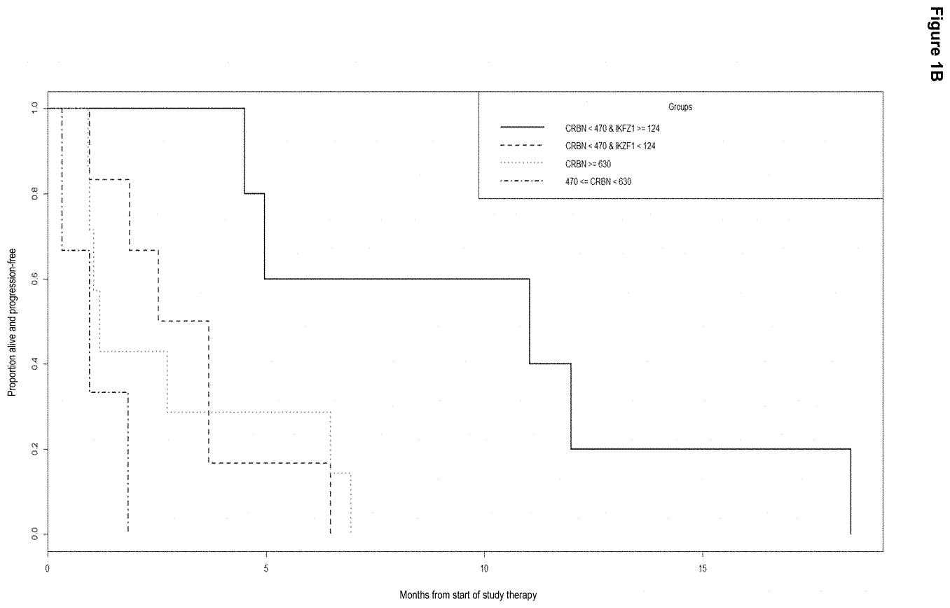

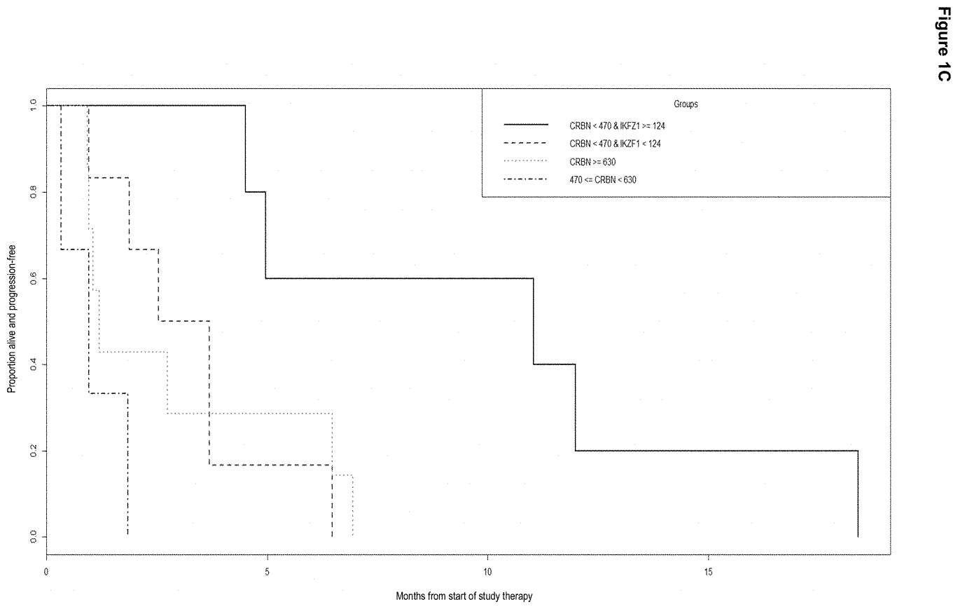

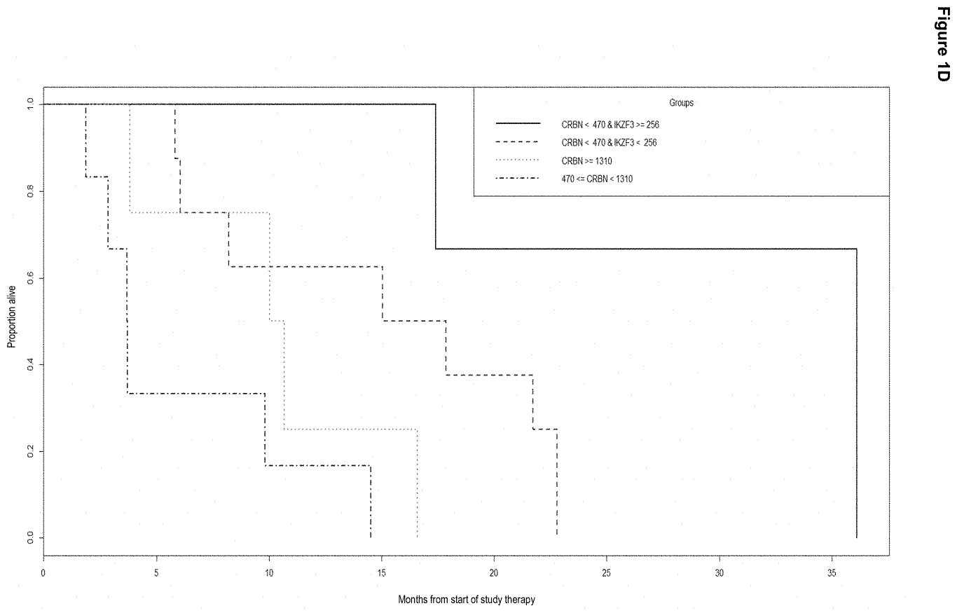

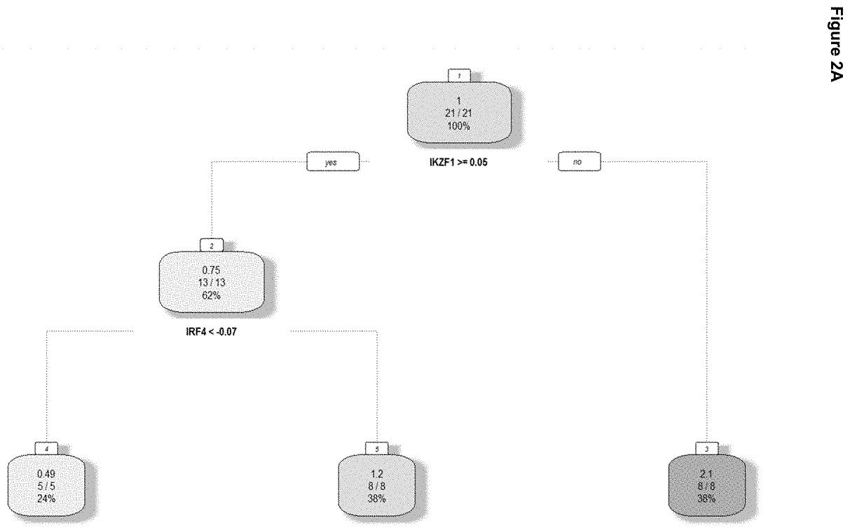

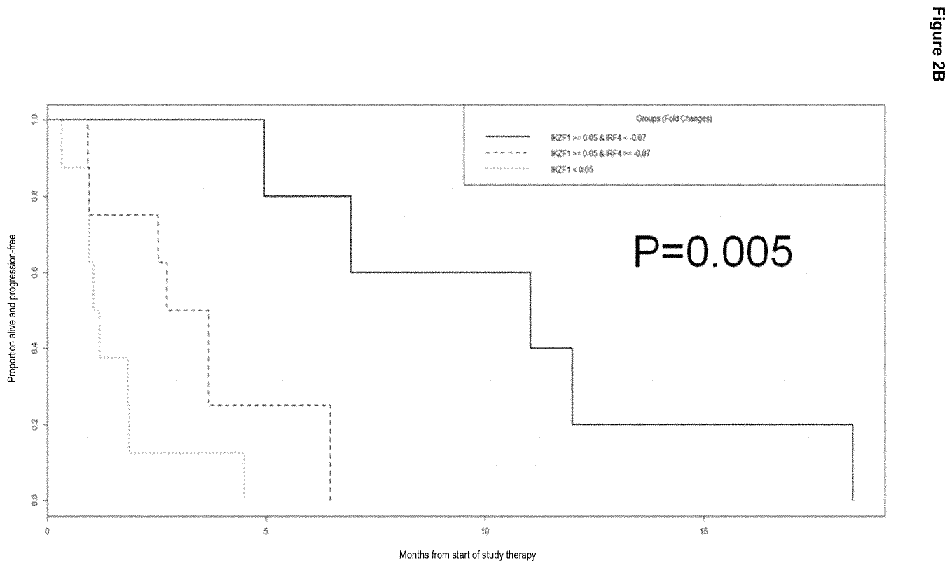

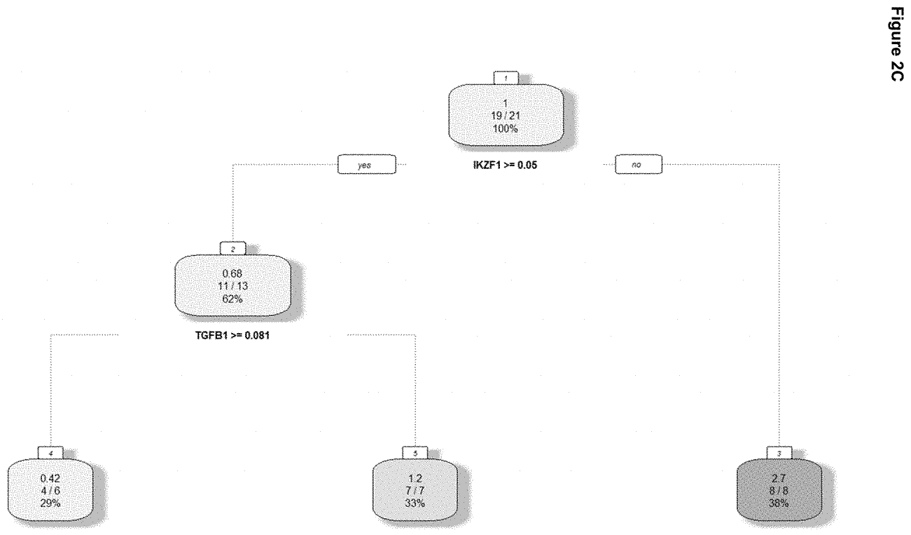

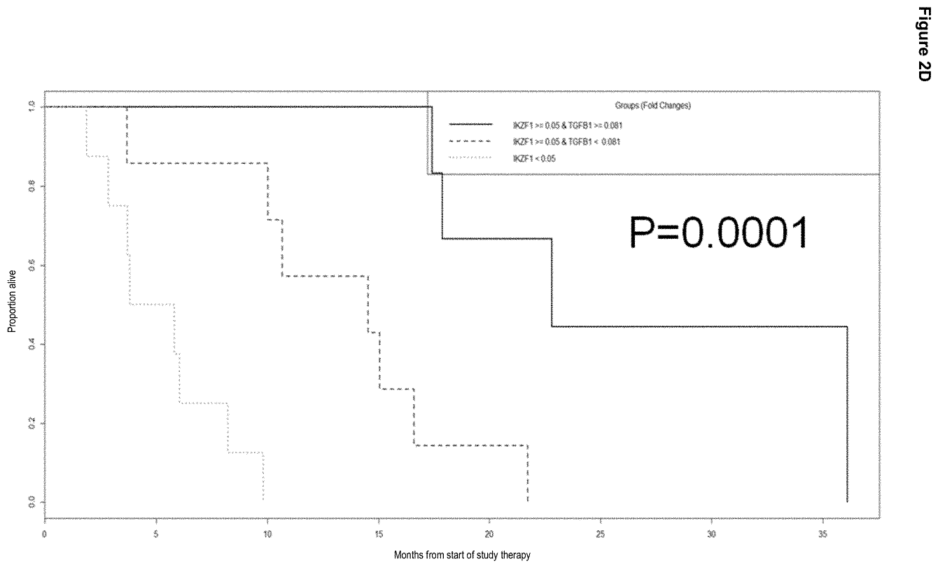

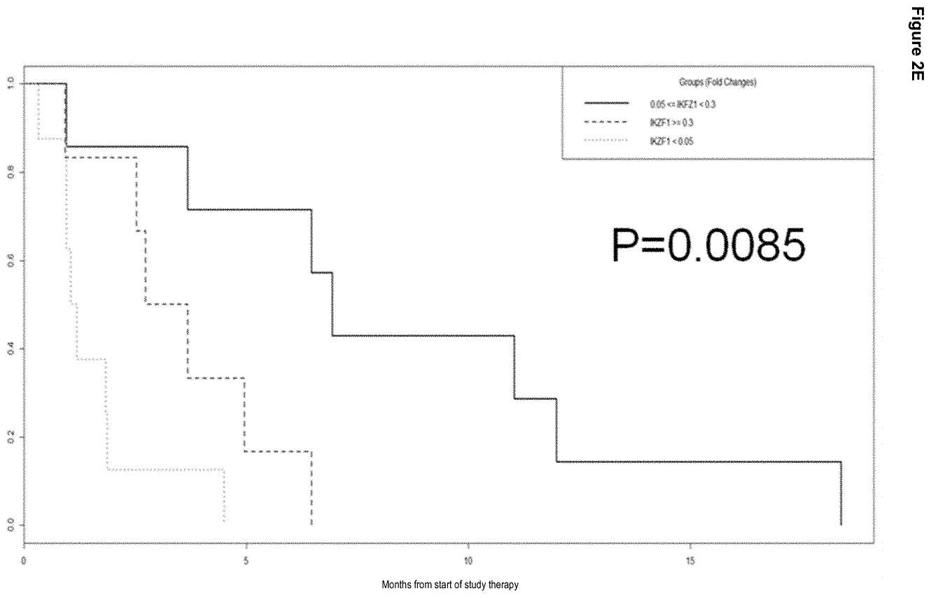

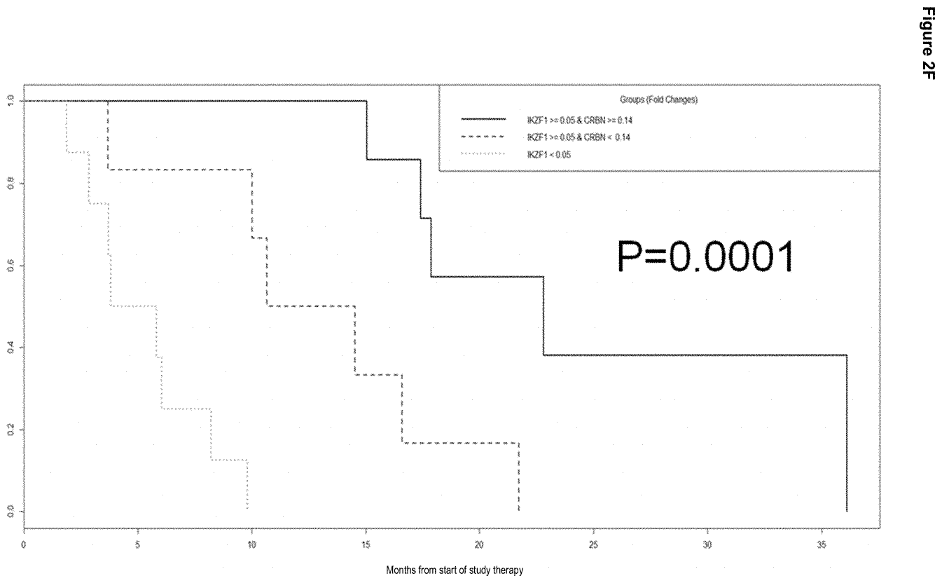

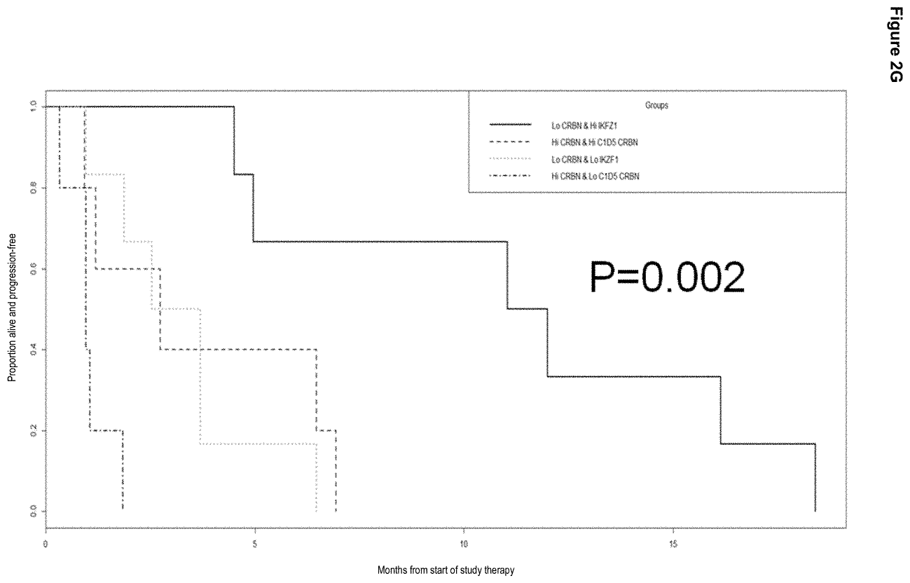

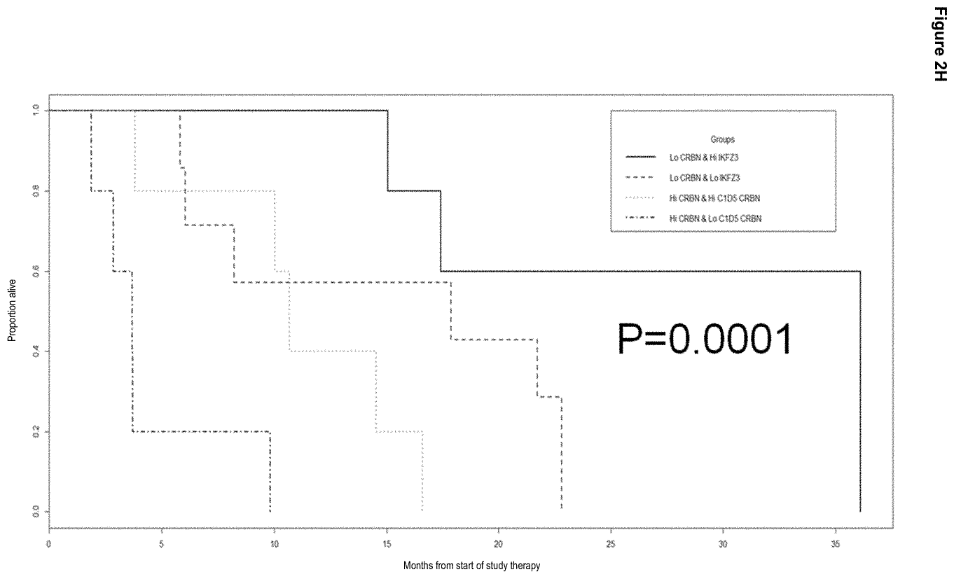

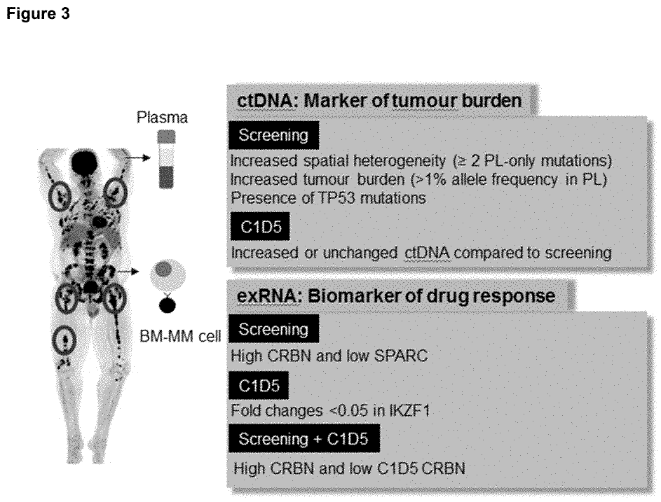

Of the 16 genes selected, a preliminary analysis to determine presence of exRNA in PL was performed that identified no detectable levels of RASD1 and BCL2L10 in a subset of patients and therefore were excluded from further testing. The levels of exRNA for 14 genes at screening and C1D5 were determined using ddPCR. The copies/ml of PL for each of these genes at the different timepoints were calculated and subjected to random forests analysis. The 5-6 exRNA with the highest variable importance measures were selected for inclusion in a single classification tree that most closely fitted the data. The best-fitting PFS tree using only top 5 exRNA indicated that intermediate levels of BSG appear to be indicative of low risk to PFS (p=0.0162). The best-fitting OS classification tree and Kaplan-Meier plot at screening indicated that low levels of CRBN coupled with high IKZF3 were associated with low risk and high CRBN levels coupled with low levels of SPARC were associated with high risk (median OS months: 36 vs 3, respectively, p=0.000003, FIGS. 1A, B). When the tree building was restricted to CRBN, IKZF1 and IKZF3, low CRBN values (<470) coupled with high IKZF1 (≥124) or high IKZF3 (≥256) seem to be associated with low PFS and OS risk, respectively (p=0.014 for PFS and p=0.005 for OS; FIGS. 1C, D). Patients with high CRBN levels at screening were associated with high risk for progression in both PFS and OS (FIGS. 1C, D). To identify if the source of exRNA is the PBMC or BM, a comparison of CRBN, IKZF1, IKZF3 and IRF4 levels at screening was analysed between responders and non-responders with a significant increase only in IKZF1 noted in PBMC of responders compared to non-responders screening samples. There were no other changes mRNA of CRBN, IKZF1, IKZF3 and IRF4 in the whole BM screening samples (data not shown).

Increased exRNA Levels as Early Markers of Response to Therapy

Alterations in levels of exRNA at C1D5 compared to screening were correlated to survival. As before, random forest analysis was utilised to select the top 5 exRNA to fit in a classification tree. Fold changes in IKFZF1 0.05 coupled with fold changes in IRF4<−0.07 or TGFB1≥0.081 were associated with a low likelihood of survival (low risk of PFS or OS), respectively and fold changes in IKZF1<0.05 was associated with high likelihood of survival (high risk risk in both PFS and OS) (p=0.0051 PFS and p=0.0001 OS, FIG. 2A-D). When the tree-building was restricted to fold changes in CRBN, IKZF1 and IKZF3, IKZF1≥0.05 is associated with low risk, both in PFS and OS (p=0.0085 and p=0.0001, respectively (FIG. 2E-F). Additionally, instead of TGFB1, patients with CRBN increase coupled with increasing IKZF1 have a better prognosis for OS (p=0.0001, FIG. 2F). There were no significant differences in either PFS or OS in patients that had an increase or decrease in levels of CRBN and IKZF1 in PBMC and in whole BM aspirates. When a combination of both screening levels and fold changes were analysed to predict biomarkers of response with the tree-building restricted to CRBN, IKZF1 and IKZF3, it was revealed that patients with high CRBN screening levels coupled with a low increase in CRBN following treatment were at the highest risk of progression while patients with low CRBN coupled with high IKZF1 or IKZF3 were at the lowest risk of progression (PFS, p=0.002 and OS, p=0.0001, FIGS. 2G, H).

Discussion:

Circulating nucleic acids have tremendous potential to be utilised as non-invasive cancer biomarkers of response to therapy. In this study, the potential utility of exRNA to predict prognosis to therapy in MM patients has been investigated. To date, this is the first study to comprehensively analyse and observe exRNA as an early biomarker of response to therapy in annotated cancer patients.

Analyses of exRNA of specific genes that are known to be regulated by the treatments received (in this example, by LEN and CC-486) were investigated. Immunomodulatory (IMiDs) drugs like LEN are known to bind to CRBN, a substrate receptor of the CRL4CRBN E3 ligase complex, increasing the affinity of CRBN for the lymphoid transcription factors IKZF1 and IKZF3, thus resulting in an increase in their ubiquitination and degradation. Random forest analysis of exRNA indicated that if CRBN is upregulated at C1D5 this suggests that the patient has a better prognosis and has likely responded to the treatment (FIG. 3). Therefore, modulation in CRBN is a critical biomarker of response to therapy.

Unlike ctDNA, it is not feasible to delineate if the source of exRNA is the MM cells and/or microenviroment, both of which can be regulated by RD and/or CC-486. Since the therapeutics utilised elicit both MM and immune cell response analyzing exRNA, which is a composite of tumour and non-tumour cells, is a suitable complementary tool. Furthermore, this type of analyses is particularly valuable when drugs that are reliant on host immune response are utilised. Discovery of biomarkers relevant to specific therapeutics can also be enabled by next-generation sequencing (NGS) of exRNA before and after treatment, thus providing a novel non-invasive approach to assess patient response and to determine distinct alterations that reflect dysregulation of not only tumour cells but also immune cells and the microenviroment.

In summary, correlative studies using exRNA undertaken with this study have provided relevant early biomarkers of response, which are readily assessable and non-invasive, to CC-486 and LEN-DEX, affording a more targeted therapy approach for patients enrolled in MM clinical trials.

1. A method for monitoring the response of an individual to a treatment for multiple myeloma, the method comprising:
providing an individual who has received a treatment for multiple myeloma, wherein the treatment includes an immunomodulatory imide (IMid) compound;
determining the level of one or more of the genes cereblon, ikaros and aiolos in a test sample of extracellular RNA (exRNA) from the individual;
comparing the level of the one or more of cereblon, ikaros and aiolos in the test sample to a control profile representative of exRNA in a multiple myeloma patient prior to treatment for multiple myeloma;
wherein an increase in the level of one or more of cereblon, ikaros and aiolos in the test sample compared to the control indicates that the individual has responded to the treatment.

2. A method for monitoring the response of an individual to a treatment for multiple myeloma, the method comprising:
providing a test sample of exRNA from an individual who has received a treatment for multiple myeloma, wherein the treatment includes an IMid;
determining the level of one or more of cereblon, ikaros and aiolos in the test sample;
providing a control profile containing data on the level of exRNA from the genes cereblon, ikaros and aiolos in the individual prior to receiving the treatment;
comparing the level of one or more of cereblon, ikaros and aiolos in the test sample to the control profile;
wherein an increase in the level of one or more of cereblon, ikaros and aiolos in the test sample compared to the control indicates that the individual has responded to the treatment.

3. A method according to claim 1 or 2, wherein the level of exRNA of interferon regulatory factor 4 (IRF4) are also determined in the test sample, wherein a decrease in the level of IRF4 in the test sample compared to the control sample indicates the individual has responded to treatment.

4. A method according to any one of claims 1 to 3, wherein the level of exRNA of transcription growth factor beta 1 (TGFβ1) are also determined in the test sample, wherein an increase in the level of TGFβ1 in the test sample compared to the control sample indicates the individual has responded to treatment.

5. A method according to any one of claims 1 to 4, wherein the test sample is obtained fewer than 20 days after commencement of the treatment for multiple myeloma.

6. A method according to claim 5, wherein the test sample is obtained fewer than 15 or 10 days after commencement of the treatment for multiple myeloma.

7. A method according to claim 5, wherein the test sample is obtained fewer than 5 days after commencement of the treatment for multiple myeloma.

8. A method according to claim 5, wherein the test sample is obtained fewer than 4 days after commencement of the treatment for multiple myeloma.

9. A method according to claim 5, wherein the test sample is obtained fewer than 3 days after commencement of the treatment for multiple myeloma.

10. A method according to claim 5, wherein the test sample is obtained fewer than 2 days after commencement of the treatment for multiple myeloma.

11. A method according to any one of claims 1 to 10, wherein the individual who has received treatment for multiple myeloma is an individual with relapsed and/or refractory multiple myeloma.

12. A method according to any one of claims 1 to 10, wherein the individual has not responded to a prior treatment.

13. A method according to claim 12, wherein the prior treatment included lenalidomide but not in combination with azacitidine or dexamethasone.

14. A method according to one of claims 1 to 13, wherein an increase in the level of cereblon indicates that the individual has responded to the treatment.

15. A method according to any one of claims 1 to 13, wherein an increase in the level of at least cereblon and ikaros indicates that the individual has responded to treatment.

16. A method according to any one of claims 1 to 13, wherein an increase in the level of cereblon, ikaros and aiolos indicates the individual has responded to treatment.

17. A method according to any one of claims 1 to 16, wherein the IMid is selected from lenalidomide, pomolidomide, thalidomide and apremilast.

18. A method according to any one of claims 1 to 17, wherein the IMid is lenalidomide.

19. A method according to any one of claims 1 to 18, wherein the treatment for multiple myeloma further includes a hypomethylating agent.

20. A method according to claim 19, wherein the hypomethylating agent includes azacitidine.

21. A method according to any one of claims 1 to 20, wherein the treatment for multiple myeloma is selected from: the combination of azacitidine and lenalidomide, or the combination of azacitidine, lenalidomide and dexamethasone.

22. A method of treating an individual for multiple myeloma, the method comprising:
providing an individual who has received a treatment for multiple myeloma, wherein the treatment includes an IMid;
determining the expression of one or more of cereblon, ikaros and aiolos in a test sample of extracellular RNA (exRNA) from the individual;
comparing the expression of the gene in the test sample to a control profile representative of exRNA in a multiple myeloma patient prior to treatment for multiple myeloma;
ceasing the treatment and commencing the individual on an alternative treatment when the expression of one or more of cereblon, ikaros and aiolos in the test sample stays the same or does not increase in response to the treatment.

23. A method of treating an individual for multiple myeloma, the method comprising:
providing an individual who has received a treatment for multiple myeloma, wherein the treatment includes an IMid;
determining the expression of one or more of cereblon, ikaros and aiolos in a test sample of extracellular RNA (exRNA) from the individual;
comparing the expression of one or more of cereblon, ikaros and aiolos in the test sample to a control profile representative of exRNA in a multiple myeloma patient prior to treatment for multiple myeloma;
continuing the treatment when the expression of one or more of cereblon, ikaros and aiolos in the test sample increases in response to the treatment.

24. A method of claim 22 or 23, wherein the step of comparing the expression of one or more of cereblon, ikaros and aiolos in the test sample to a control profile, and the step of ceasing or continuing the treatment is performed within 20 days of the commencement of treatment.

25. A method of claim 24 wherein the step of comparing is done fewer than 15, fewer than 10, fewer than 5 or fewer than 3 days following commencement of the treatment.

26. A method of claim 22, wherein commencing the individual on an alternative treatment includes supplementing the treatment with one or more additional drugs.

27. A method of claim 22, wherein commencing the individual on an alternative treatment includes replacing the treatment with one or more alternative drugs.

28. The method according to claim 26 or 27, wherein the additional drugs or alternative drugs for treatment for multiple myeloma are selected from the group consisting of: dexamethasone, Cyclophosphamide, Thalidomide, Pomalidomide, Etoposide, Cisplatin, Ixazomib, Bortezomib, Vemurafinib, Venetoclax, Trametinib, Panobinostat, Vorinostat, Azacytidine, Daratumumab, Pembrolizumab, Nivolumumab, Durvalumab or autologous stem cell transplant (ASCT), or combinations thereof.

29. The method according to any one of the preceding claims wherein the test sample of exRNA is any biological sample obtained from the individual that contains exRNA selected from: peripheral blood, saliva, breast milk, urine, semen, menstrual blood, and vaginal fluid.

30. The method according to claim 29, wherein the biological sample is peripheral blood.

31. The method according to any one of the preceding claims, wherein the control profile is any biological sample obtained from an individual that has multiple myeloma or has received a treatment for multiple myeloma, wherein the biological sample contains exRNA.

32. The method according to any one of the preceding claims, wherein the control biological sample containing exRNA is obtained from the individual prior to receiving treatment for multiple myeloma.

33. The method according to any one of claims 1 to 32, wherein the control profile is obtained from a database comprising exRNA levels in a biological sample from one or more multiple myeloma patients, obtained prior to the patients receiving treatment for multiple myeloma.

34. The method according to any one of the preceding claims wherein the control profile is obtained 1, 2, 5, 10, 20, 30 or more days prior to the individual receiving treatment for multiple myeloma.

35. A method for determining the likelihood that an individual will respond to a treatment for multiple myeloma, wherein the treatment comprises an immunomodulatory imide (IMid) compound, the method comprising:
determining the expression of ikaros in a test sample comprising exRNA from an individual who has been diagnosed with or is suspected of having multiple myeloma;
wherein the presence of expression of ikaros in the test sample indicates that the patient will respond to the treatment; and
wherein the absence of expression of ikaros in the test sample indicates that the individual will not respond to the treatment.

36. A method for determining the likelihood that an individual will respond to a treatment for multiple myeloma, wherein the treatment comprises an immunomodulatory imide (IMid) compound and a hypomethylating agent, the method comprising:
determining the expression of cereblon in a test sample of exRNA from an individual who has been diagnosed with or is suspected of having multiple myeloma;
wherein a high level of expression of cereblon in the test sample indicates at the patient will not respond to the treatment, and
wherein a low level of expression of cereblon in the test sample indicates that the individual will respond to the treatment.

37. A method according to claim 36, wherein the method further includes determining the expression one or more of ikaros and aiolos, and wherein when the individual has a low level of expression of cereblon, coupled with high levels of ikaros and aiolos prior to treatment, is indicative that the individual will likely respond to treatment.

38. A method according to claim 36 or 37, wherein the method further includes determining the expression of SPARC, and wherein the individual has a low level of expression of cereblon, coupled with a high level of SPARC prior to treatment, indicates that the individual will likely respond to the treatment.

39. A method according to any one of claims 36 to 38, wherein a high level of expression of cereblon refers to copy numbers of the cereblon transcript in a sample of exRNA of at least 400, at least 450, preferably more than 470 copies/mL.

40. A method according to any one of claims 36 to 39, wherein a low level of expression of cereblon refers to copy numbers of the cereblon transcript in a sample of exRNA of less than 400, less than 300, or less than 100 copies/mL.

41. A method according to claim 35 or 37, wherein a high level of expression of ikaros refers to copy numbers of the ikaros transcript in a sample of exRNA of at least 80, at least 100, preferably more than 120 copies/mL.

42. A method according to claim 35 or 37, wherein a low level of expression of ikaros refers to copy numbers of the ikaros transcript in a sample of exRNA of less than 80, less than 50, or less than 20 copies/mL.

43. A method according to claim 35 or 37, wherein a high level of expression of aiolos refers to copy numbers of the aiolos transcript in a sample of exRNA of at least 200, at least 240, preferably more than 250 copies/m.

44. A method according to claim 35 or 37, wherein a low level of expression of aiolos refers to copy numbers of the aiolos transcript in a sample of exRNA of less than 200, less than 100, or less than 50 copies/mL plasma.

45. A method according to any one of claims 35 to 44, wherein the IMid is selected from lenalidomide, pomolidomide, thalidomide and apremilast.

46. A kit for use in monitoring the response of an individual to a treatment for multiple myeloma, the kit comprising:
a means for detecting exRNA levels corresponding to one or more genes;
reagents for isolating or extracting exRNA from a peripheral blood sample of an individual.

47. A kit according to claim 46, further comprising written instructions for use of the kit in a method according to any one of claims 1 to 45.
